(19) United States
(12) Patent Application Publication
(10) Pub. No.: US 2023/0365678 A1
(43) Pub. Date: Nov. 16, 2023

(54) DOSING REGIMENS OF ANTI-ILT4 ANTIBODY OR ITS COMBINATION WITH ANTI-PD-1 ANTIBODY FOR TREATING CANCER

(71) Applicant: Merck Sharp & Dohme LLC, Rahway, NJ (US)

(72) Inventors: Anson K. Abraham, Furlong, PA (US); Rachel A. Altura, Westerly, RI (US); Douglas C. Wilson, San Francisco, CA (US)

(73) Assignee: Merck Sharp & Dohme LLC, Rahway, NJ (US)

(21) Appl. No.: 18/245,590

(22) PCT Filed: Sep. 15, 2021
(86) PCT No.: PCT/US2021/050365

§ 371 Date: Mar. 16, 2023

Related U.S. Application Data
(60) Provisional application No. 63/079,976, filed on Sep. 17, 2020.

(51) Int. Cl. C07K 16/28; A61P 35/00

(52) U.S. Cl. CPC C07K 16/2803; A61P 35/00; C07K 16/2818; A61K 2039/545

(57) ABSTRACT

Provided herein are dosing regimens of an anti-immunoglobulin-like transcript 4 (anti-ILT4) antibody for treating cancer. Further provided are dosing regimens for treating cancer using a combination of an anti-ILT4 antibody and another agent (e.g., a PD-1 antagonist, such as an anti-PD-1 antibody or an anti-PD-L 1 antibody).

### FIELD OF THE INVENTION

The present invention relates to dosing regimens of an anti-immunoglobulin-like transcript 4 (anti-ILT4) antibody for treating cancer. It also relates to dosing regimens for treating cancer using a combination of an anti-ILT4 antibody and another agent (e.g., a PD-1 antagonist, such as an anti-PD-1 antibody or an anti-PD-L1 antibody).

### CROSS-REFERENCE TO RELATED APPLICATIONS

This application claims the benefit of U.S. Provisional Application No. 63/079,976, filed Sep. 17, 2020, the contents of which are hereby incorporated by reference in their entirety.

### REFERENCE TO SEQUENCE LISTING SUBMITTED ELECTRONICALLY

The sequence listing of the present application is submitted electronically via EFS-Web as an ASCII formatted sequence listing with a file name 25104WOPCT-SEQLIST-13AUG.2021.txt, creation date of Aug. 13, 2021, and a size of 19.2 kb. This sequence listing submitted via EFS-Web is part of the specification and is herein incorporated by reference in its entirety.

### BACKGROUND OF THE INVENTION

PD-1 is recognized as an important player in immune regulation and the maintenance of peripheral tolerance. Immune checkpoint therapies targeting PD-1 or its ligand (e.g., PD-L1) have resulted in groundbreaking improvements in clinical response in multiple human cancer types (Brahmer et al., N Engl J Med, 366: 2455-2465 (2012); Garon et al., N Engl J Med, 372:2018-2028 (2015); Hamid et al., N Engl J Med, 369:134-144 (2013); Robert et al., Lancet, 384:1109-1117 (2014); Robert et al., N Engl J Med, 372: 2521-2532 (2015); Robert et al., N Engl J Med, 372:320-330 (2015); Topalian et al., N Engl J Med, 366:2443-2454 (2012); Topalian et al., J Clin Oncol, 32:1020-1030 (2014); Wolchok et al., N Engl J Med, 369:122-133 (2013)). Immune therapies targeting the PD-1 axis include monoclonal antibodies directed to the PD-1 receptor (e.g., KEYTRUDA® (pembrolizumab), Merck and Co., Inc., Kenilworth, NJ; OPDIVO® (nivolumab), Bristol-Myers Squibb Company, Princeton, NJ) and those that bind to the PD-L1 ligand (e.g., TECENTRIQ® (atezolizumab), Genentech, San Francisco, CA). Another common strategy used by tumor cells to escape innate and adaptive immune response is associated with aberrant expression of human leukocyte antigen (HLA)-G (Curigliano et al. Clin Cancer Res. 2013 and Gonzalez et al. Crit Rev Clin Lab Sci. 2012). HLA-G can directly inhibit immune cell function through receptor binding and/or through trogocytosis and impairment of chemotaxis (Morandi et al. Cytokine Growth Factor Review. 2014 and Lin et al. Mol Med. 2015). Antibody-mediated blockade of HLA-G function in transgenic mouse models has been shown to inhibit tumor development and block expansion of myeloid-derived suppressor cells (MDSC) (Loumange et al. Int J Cancer. 2014, Lin et al. Hum Immunol. 2013, and Agaugue et al. Blood. 2011). HLA-G binding to ILT4 can directly inhibit the function of monocytes, dendritic cells, and neutrophils, thus impairing the innate immune anti-tumor response. Accordingly, ILT4 blockade was predicted to relieve suppression of tolerogenic myeloid cells in the tumor microenvironment, and this has been supported by experimental evidence (Chen et al., J. Clin. Invest. 2018, 128(12):5647-5662).

Selecting a dosing regimen for an anti-ILT4 antibody monotherapy or combination therapy with another agent (e.g., a PD-1 antagonist, such as an anti-PD-1 antibody or an anti-PD-L1 antibody) depends on many factors, including the serum or tissue turnover rate of the entity, the level of symptoms, the immunogenicity of the entity, anti-drug antibody endpoints and the accessibility of the target cells, tissue or organ in the individual being treated, as well as safety. Formation of anti-drug antibodies can potentially confound drug exposures at therapeutic doses, and prime for subsequent infusion-related toxicities. In addition, anti-ILT4 antibody and/or anti-PD-1/anti-PD-L1 antibody treatment can result in immune stimulation and potential cytokine release that affects safety. Thus, there is an unmet need to identify a safe and effective dosing regimen for an anti-ILT4 antibody, either alone or in combination with another agent (e.g., a PD-1 antagonist, such as an anti-PD-1 antibody or an anti-PD-L1 antibody), in treating cancer.

### SUMMARY OF THE INVENTION

The present disclosure provides methods of treating cancer in a patient comprising administering to the patient a certain dosage of an anti-ILT4 antibody, either alone or in combination with a certain dosage of another agent (e.g., a PD-1 antagonist, such as an anti-PD-1 antibody or an anti-PD-L1 antibody). Also provided are pharmaceutical compositions comprising a certain dosage of an anti-ILT4 antibody and a certain dosage of another agent (e.g., a PD-1 antagonist, such as an anti-PD-1 antibody or an anti-PD-L1 antibody), as well as kits comprising a certain dosage of an anti-ILT4 antibody and a certain dosage of another agent (e.g., a PD-1 antagonist, such as an anti-PD-1 antibody or an anti-PD-L1 antibody).

In one aspect, provided herein is a method for treating cancer in a patient comprising administering to the patient 3-1600 mg of an anti-ILT4 antibody, wherein the anti-ILT4 antibody comprises: (a) a light chain variable domain comprising CDR-L1 of SEQ ID NO:11, CDR-L2 of SEQ ID NO:21, and CDR-L3 of SEQ ID NO:13; and (b) a heavy chain variable domain comprising CDR-H1 of SEQ ID NO:16, CDR-H2 of SEQ ID NO:22, and CDR-H3 of SEQ ID NO:18.

In certain embodiments, the anti-ILT4 antibody comprises: (a) a light chain variable domain comprising CDR-L1 of SEQ ID NO:11, CDR-L2 of SEQ ID NO:12, and CDR-L3 of SEQ ID NO:13; and (b) a heavy chain variable domain comprising CDR-H1 of SEQ ID NO:16, CDR-L2 of SEQ ID NO:17, and CDR-H3 of SEQ ID NO:18.

In some embodiments, the anti-ILT4 antibody is administered to the patient via intravenous infusion.

In one embodiment, the patient is administered 30 mg of the anti-ILT4 antibody.

In another embodiment, the patient is administered 100 mg of the anti-ILT4 antibody.

In certain embodiments, the patient is administered 300-1600 mg of the anti-ILT4 antibody.

In one embodiment, the patient is administered 300 mg of the anti-ILT4 antibody.

In another embodiment, the patient is administered 800 mg of the anti-ILT4 antibody.

In yet another embodiment, the patient is administered 1600 mg of the anti-ILT4 antibody.

In some embodiments, the patient is administered the anti-ILT4 antibody on Day 1 and then once approximately every three weeks thereafter.

In certain embodiments, the anti-ILT4 antibody or antigen binding fragment thereof comprises a heavy chain variable region of SEQ ID NO:19 and a light chain variable region of SEQ ID NO:14.

In other embodiments, the anti-ILT4 antibody or antigen binding fragment thereof comprises a heavy chain of SEQ ID NO:20 and a light chain of SEQ ID NO:15.

In some embodiments, the anti-ILT4 antibody is co-administered with a PD-1 antagonist.

In other embodiments, the anti-ILT4 antibody is co-formulated with a PD-1 antagonist.

In certain embodiments, the PD-1 antagonist is an anti-PD-1 antibody or antigen binding fragment thereof.

In other embodiments, the PD-1 antagonist is an anti-PD-L1 antibody or antigen binding fragment thereof.

In some embodiments, the anti-PD-1 antibody or antigen binding fragment thereof comprises: (a) a light chain variable domain comprising CDR-L1 of SEQ ID NO:1, CDR-L2 of SEQ ID NO:2, and CDR-L3 of SEQ ID NO:3; and (b) a heavy chain variable domain comprising CDR-H1 of SEQ ID NO:6, CDR-L2 of SEQ ID NO:7, and CDR-H3 of SEQ ID NO:8.

In other embodiments, the anti-PD-1 antibody or antigen binding fragment thereof comprises a heavy chain variable region of SEQ ID NO:9 and a light chain variable region of SEQ ID NO:4.

In yet other embodiments, the anti-PD-1 antibody or antigen binding fragment thereof comprises a heavy chain of SEQ ID NO:10 and a light chain of SEQ ID NO:5.

In one embodiment, the anti-PD-1 antibody is pembrolizumab.

In another embodiment, the anti-PD-1 antibody is a pembrolizumab variant.

In yet another embodiment, the anti-PD-1 antibody is nivolumab or a nivolumab variant.

In still another embodiment, the anti-PD-1 antibody is cemiplimab or a cemiplimab variant.

In one embodiment, the anti-PD-L1 antibody is atezolizumab or an atezolizumab variant.

In another embodiment, the anti-PD-L1 antibody is durvalumab or a durvalumab variant.

In yet another embodiment, the anti-PD-L1 antibody is avelumab or an avelumab variant.

In certain embodiments of various methods provided herein, the anti-PD-1 antibody is pembrolizumab or a pembrolizumab variant administered at 200 mg via intravenous infusion on Day 1 and then once every about three weeks thereafter.

In some embodiments, the anti-PD-1 antibody is pembrolizumab or a pembrolizumab variant administered at 400 mg via intravenous infusion on Day 1 and then once every about six weeks thereafter.

In other embodiments, the anti-PD-1 antibody is nivolumab or a nivolumab variant administered at 240 mg via intravenous infusion on Day 1 and then once every about two weeks thereafter.

In yet other embodiments, the anti-PD-1 antibody is nivolumab or a nivolumab variant administered at 480 mg via intravenous infusion on Day 1 and then once every about four weeks thereafter.

In still other embodiments, the anti-PD-1 antibody is cemiplimab or a cemiplimab variant administered at 350 mg via intravenous infusion on Day 1 and then once every about three weeks thereafter.

In some embodiments, the anti-PD-L1 antibody is atezolizumab or an atezolizumab variant administered at 840 mg via intravenous infusion on Day 1 and then once every about two weeks thereafter.

In other embodiments, the anti-PD-L1 antibody is atezolizumab or an atezolizumab variant administered at 1200 mg via intravenous infusion on Day 1 and then once every about three weeks thereafter.

In yet other embodiments, the anti-PD-L1 antibody is atezolizumab or an atezolizumab variant administered at 1680 mg via intravenous infusion on Day 1 and then once every about four weeks thereafter.

In still other embodiments, the anti-PD-L1 antibody is durvalumab or a durvalumab variant administered at 1500 mg via intravenous infusion on Day 1 and then once every about three weeks thereafter.

In yet still embodiments, the anti-PD-L1 antibody is avelumab or an avelumab variant administered at 800 mg via intravenous infusion on Day 1 and then once every about two weeks thereafter.

In some embodiments, provided is a method for treating cancer in a patient comprising administering to the patient 3-1600 mg of an anti-ILT4 antibody and 200-400 mg of an anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3; and the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In other embodiments, the anti-PD-1 antibody comprises a heavy chain variable region of SEQ ID NO:9 and a light chain variable region of SEQ ID NO: 4; and the anti-ILT4 antibody comprises a heavy chain variable region of SEQ ID NO:19 and a light chain variable region of SEQ ID NO:14.

In yet other embodiments, the anti-PD-1 antibody comprises a heavy chain of SEQ ID NO:10 and a light chain of SEQ ID NO: 5; and the anti-ILT4 antibody comprises a heavy chain of SEQ ID NO:20 and a light chain of SEQ ID NO:15.

In certain embodiments, 200 mg of the anti-PD-1 antibody is administered to the patient via intravenous infusion on Day 1 and then once approximately every three weeks thereafter, and 300 mg of the anti-ILT4 antibody is administered to the patient via intravenous infusion on Day 1 and then once approximately every three weeks thereafter.

In some embodiments, 400 mg of the anti-PD-1 antibody is administered to the patient via intravenous infusion on Day 1 and then once every six weeks thereafter, and 300 mg of the anti-ILT4 antibody is administered to the patient via intravenous infusion on Day 1 and then once approximately every three weeks thereafter.

In other embodiments, 200 mg of the anti-PD-1 antibody is administered to the patient via intravenous infusion on Day 1 and then once approximately every three weeks thereafter, and 800 mg of the anti-ILT4 antibody is administered to the patient via intravenous infusion on Day 1 and then once approximately every three weeks thereafter.

In yet other embodiments, 400 mg of the anti-PD-1 antibody is administered to the patient via intravenous infusion on Day 1 and then once every about six weeks thereafter, and 800 mg of the anti-ILT4 antibody is administered to the patient via intravenous infusion on Day 1 and then once approximately every three weeks thereafter.

In still other embodiments, 200 mg the anti-PD-1 antibody is administered to the patient via intravenous infusion on Day 1 and then once approximately every three weeks thereafter, and 1600 mg of the anti-ILT4 antibody is administered to the patient via intravenous infusion on Day 1 and then once approximately every three weeks thereafter.

In yet still other embodiments, 400 mg of the anti-PD-1 antibody is administered to the patient via intravenous infusion on Day 1 and then once every about six weeks thereafter, and 1600 mg of the anti-ILT4 antibody is administered to the patient via intravenous infusion on Day 1 and then once approximately every three weeks thereafter.

In certain embodiments of various methods disclosed herein, the anti-PD-1 antibody and the anti-ILT4 antibody are co-formulated in one pharmaceutical composition. In one embodiment, 200 mg of anti-PD-1 antibody is co-formulated with 300-1600 mg of anti-ILT4 antibody. In another embodiment, 200 mg of anti-PD-1 antibody is co-formulated with 300 mg, 800 mg, or 1600 mg of anti-ILT4 antibody. In one embodiment, 200 mg of anti-PD-1 antibody is co-formulated with 300 mg of anti-ILT4 antibody. In another embodiment, 200 mg of anti-PD-1 antibody is co-formulated with 800 mg of anti-ILT4 antibody. In yet another embodiment, 200 mg of anti-PD-1 antibody is co-formulated with 1600 mg of anti-ILT4 antibody.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., NSCLC), pleural mesothelioma, gastric cancer, colorectal cancer, cervical cancer, synovial sarcoma, head and neck cancer, squamous cell carcinoma, lymphoma (e.g., diffuse large B-cell lymphoma (DLBCL) or non-Hodgkin lymphoma (NHL)), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelofibrosis, soft tissue sarcoma, thyroid cancer, endometrial cancer, and carcinoid cancer.

In some embodiments, the cancer is selected from the group consisting of: melanoma, lung cancer, head and neck cancer, bladder cancer, breast cancer, gastrointestinal cancer, multiple myeloma, hepatocellular cancer, merkel cell carcinoma, cutaneous squamous cell carcinoma, lymphoma, renal cancer, mesothelioma, ovarian cancer, esophageal cancer, anal cancer, biliary tract cancer, colorectal cancer, endometrial cancer, cervical cancer, thyroid cancer, salivary cancer, prostate cancer (e.g. hormone refractory prostate adenocarcinoma), pancreatic cancer, colon cancer, liver cancer, thyroid cancer, glioblastoma, glioma, and other neoplastic malignancies.

In some embodiments the lung cancer in non-small cell lung cancer.

In alternate embodiments, the lung cancer is small-cell lung cancer.

In some embodiments, the lymphoma is Hodgkin lymphoma.

In other embodiments, the lymphoma is non-Hodgkin lymphoma. In particular embodiments, the lymphoma is primary mediastinal large B-cell lymphoma (PMBCL). In some embodiments, the lymphoma is diffuse large B-cell lymphoma (DLBCL).

In some embodiments, the breast cancer is triple negative breast cancer.

In further embodiments, the breast cancer is ER+/HER2− breast cancer.

In some embodiments, the bladder cancer is urothelial cancer.

In some embodiments, the head and neck cancer is nasopharyngeal cancer. In some embodiments, the cancer is thyroid cancer. In other embodiments, the cancer is salivary cancer.

In other embodiments, the cancer is squamous cell carcinoma of the head and neck.

In some embodiments, the cancer is metastatic colorectal cancer with high levels of microsatellite instability (MSI-H).

In some embodiments, the cancer is a solid tumor with a high level of microsatellite instability (MSI-H).

In certain embodiments, the cancer is metastatic. In some embodiments, the cancer is relapsed. In other embodiments, the cancer is refractory. In yet other embodiments, the cancer is relapsed and refractory.

In some embodiments of various methods disclosed herein, the cancer is head and neck squamous cell cancer (HNSCC), gastric cancer, pancreatic cancer, glioblastoma (GBM), renal cell carcinoma (RCC), or non-small cell lung cancer (NSCLC). In one embodiment, the cancer is HNSCC. In another embodiment, the cancer is gastric cancer. In yet another embodiment, the cancer is pancreatic cancer. In still another embodiment, the cancer is GBM. In another embodiment, the cancer is RCC. In yet still another embodiment, the cancer is NSCLC.

In another aspect, provided herein is a pharmaceutical composition comprising 200-400 mg of an anti-PD-1 antibody or variant thereof, 3-1600 mg of an anti-ILT4 antibody or variant thereof, and a pharmaceutically acceptable excipient, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment of the compositions herein, the amount of anti-ILT4 antibody is 100 mg. In another embodiment, the amount of anti-ILT4 antibody is 300 mg. In yet another embodiment, the amount of anti-ILT4 antibody is 800 mg. In still another embodiment, the amount of anti-ILT4 antibody is 1600 mg. In yet still another embodiment, the amount of anti-PD-1 antibody is 200 mg.

In one embodiment, the pharmaceutical composition comprises 200 mg pembrolizumab or pembrolizumab variant, 100 mg of anti-ILT4 antibody or variant thereof, and a pharmaceutically acceptable excipient, wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, the pharmaceutical composition comprises 200 mg pembrolizumab or pembrolizumab variant, 300 mg of anti-ILT4 antibody or variant thereof, and a pharmaceutically acceptable excipient, wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In another embodiment, the pharmaceutical composition comprises 200 mg pembrolizumab or pembrolizumab variant, 800 mg of anti-ILT4 antibody or variant thereof, and a pharmaceutically acceptable excipient, wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO: 11, CDR-L2 of SEQ ID NO:12, and CDR-L3 of SEQ ID NO:13.

In yet another embodiment, the pharmaceutical composition comprises 200 mg pembrolizumab or pembrolizumab variant, 1600 mg of anti-ILT4 antibody or variant thereof, and a pharmaceutically acceptable excipient, wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO: 11, CDR-L2 of SEQ ID NO:12, and CDR-L3 of SEQ ID NO:13.

In some embodiments, provided herein is a pharmaceutical composition comprising 200-400 mg of an anti-PD-1 antibody or variant thereof, 3-1600 mg of an anti-ILT4 antibody or variant thereof, and a pharmaceutically acceptable excipient, wherein the anti-PD-1 antibody comprises a heavy chain variable region of SEQ ID NO:9 and a light chain variable region of SEQ ID NO:4, and wherein the anti-ILT4 antibody comprises a heavy chain variable region of SEQ ID NO: 19 and a light chain variable region of SEQ ID NO: 14.

In some embodiments, the pharmaceutical composition comprises 200 mg pembrolizumab or pembrolizumab variant, 300-1600 mg of anti-ILT4 antibody or variant thereof, and a pharmaceutically acceptable excipient, wherein the anti-ILT4 antibody comprises a heavy chain variable region of SEQ ID NO: 19 and a light chain variable region of SEQ ID NO:14.

In one embodiment, the pharmaceutical composition comprises 200 mg pembrolizumab or pembrolizumab variant, 300 mg of anti-ILT4 antibody or variant thereof, and a pharmaceutically acceptable excipient, wherein the anti-ILT4 antibody comprises a heavy chain variable region of SEQ ID NO: 19 and a light chain variable region of SEQ ID NO:14.

In another embodiment, the pharmaceutical composition comprises 200 mg pembrolizumab or pembrolizumab variant, 800 mg of anti-ILT4 antibody or variant thereof, and a pharmaceutically acceptable excipient, wherein the anti-ILT4 antibody comprises a heavy chain variable region of SEQ ID NO: 19 and a light chain variable region of SEQ ID NO:14.

In yet another embodiment, the pharmaceutical composition comprises 200 mg pembrolizumab or pembrolizumab variant, 1600 mg of anti-ILT4 antibody or variant thereof, and a pharmaceutically acceptable excipient, wherein the anti-ILT4 antibody comprises a heavy chain variable region of SEQ ID NO:19 and a light chain variable region of SEQ ID NO:14.

In some embodiments, provided herein is a pharmaceutical composition comprising 200-400 mg of an anti-PD-1 antibody or variant thereof, 3-1600 mg of an anti-ILT4 antibody or variant thereof, and a pharmaceutically acceptable excipient, wherein the anti-PD-1 antibody comprises a heavy chain of SEQ ID NO:10 and a light chain of SEQ ID NO:5, and wherein the anti-ILT4 antibody comprises a heavy chain of SEQ ID NO:20 and a light chain of SEQ ID NO:15.

In other embodiments, the pharmaceutical composition comprises 200 mg pembrolizumab or pembrolizumab variant, 300-1600 mg of anti-ILT4 antibody or variant thereof, and a pharmaceutically acceptable excipient, wherein the anti-ILT4 antibody comprises a heavy chain of SEQ ID NO:20 and a light chain of SEQ ID NO: 15.

In one embodiment, the pharmaceutical composition comprises 200 mg pembrolizumab or pembrolizumab variant, 300 mg of anti-ILT4 antibody or variant thereof, and a pharmaceutically acceptable excipient, wherein the anti-ILT4 antibody comprises a heavy chain of SEQ ID NO:20 and a light chain of SEQ ID NO:15.

In another embodiment, the pharmaceutical composition comprises 200 mg pembrolizumab or pembrolizumab variant, 800 mg of anti-ILT4 antibody or variant thereof, and a pharmaceutically acceptable excipient, wherein the anti-ILT4 antibody comprises a heavy chain of SEQ ID NO:20 and a light chain of SEQ ID NO:15.

In yet another embodiment, the pharmaceutical composition comprises 200 mg pembrolizumab or pembrolizumab variant, 1600 mg of anti-ILT4 antibody or variant thereof, and a pharmaceutically acceptable excipient, wherein the anti-ILT4 antibody comprises a heavy chain of SEQ ID NO:20 and a light chain of SEQ ID NO: 15.

In yet another aspect, provided herein is a kit for treating cancer comprising 200-400 mg anti-PD-1 antibody or variant thereof and 3-1600 mg of anti-ILT4 antibody or variant thereof, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment of the kit of the invention, the amount of anti-ILT4 antibody is 100 mg. In another embodiment, the amount of anti-ILT4 antibody is 300 mg. In yet another embodiment, the amount of anti-ILT4 antibody is 800 mg. In still another embodiment, the amount of anti-ILT4 antibody is 1600 mg. In yet still another embodiment, the amount of anti-PD-1 antibody is 200 mg.

In one embodiment, the kit comprises 200 mg pembrolizumab or pembrolizumab variant and 3-1600 mg of anti-ILT4 antibody or variant thereof, wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, the kit comprises 200 mg pembrolizumab or pembrolizumab variant and 300 mg of anti-ILT4 antibody or variant thereof, wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In another embodiment, the kit comprises 200 mg pembrolizumab or pembrolizumab variant and 800 mg of anti-ILT4 antibody or variant thereof, wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In yet another embodiment, the kit comprises 200 mg pembrolizumab or pembrolizumab variant and 1600 mg of anti-ILT4 antibody or variant thereof, wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In some embodiments, the kit comprises 200-400 mg anti-PD-1 antibody or variant thereof and 3-1600 mg of anti-ILT4 antibody or variant thereof, wherein the anti-PD-1 antibody comprises a heavy chain variable region of SEQ ID NO:9 and a light chain variable region of SEQ ID NO:4, and wherein the anti-ILT4 antibody comprises a heavy chain variable region of SEQ ID NO: 19 and a light chain variable region of SEQ ID NO:14.

In one embodiment, the kit comprises 200 mg pembrolizumab or pembrolizumab variant and 300 mg of anti-ILT4 antibody or variant thereof, wherein the anti-ILT4 antibody comprises a heavy chain variable region of SEQ ID NO: 19 and a light chain variable region of SEQ ID NO:14.

In another embodiment, the kit comprises 200 mg pembrolizumab or pembrolizumab variant and 800 mg of anti-ILT4 antibody or variant thereof, wherein the anti-ILT4 antibody comprises a heavy chain variable region of SEQ ID NO: 19 and a light chain variable region of SEQ ID NO: 14.

In yet another embodiment, the kit comprises 200 mg pembrolizumab or pembrolizumab variant and 1600 mg of anti-ILT4 antibody or variant thereof, wherein the anti-ILT4 antibody comprises a heavy chain variable region of SEQ ID NO: 19 and a light chain variable region of SEQ ID NO: 14.

In other embodiments, the kit comprises 200-400 mg of anti-PD-1 antibody or variant and 3-1600 mg of anti-ILT4 antibody or variant thereof, wherein the anti-PD-1 antibody comprises a heavy chain of SEQ ID NO:10 and a light chain of SEQ ID NO:5, and wherein the anti-ILT4 antibody comprises a heavy chain of SEQ ID NO:20 and a light chain of SEQ ID NO:15.

In one embodiment, the kit comprises 200 mg pembrolizumab or pembrolizumab variant and 300 mg of anti-ILT4 antibody or variant thereof, wherein the anti-ILT4 antibody comprises a heavy chain of SEQ ID NO:20 and a light chain of SEQ ID NO: 15.

In another embodiment, the kit comprises 200 mg pembrolizumab or pembrolizumab variant and 800 mg of anti-ILT4 antibody or variant thereof, wherein the anti-ILT4 antibody comprises a heavy chain of SEQ ID NO:20 and a light chain of SEQ ID NO: 15.

In yet another embodiment, the kit comprises 200 mg pembrolizumab or pembrolizumab variant and 1600 mg of anti-ILT4 antibody or variant thereof, wherein the anti-ILT4 antibody comprises a heavy chain of SEQ ID NO:20 and a light chain of SEQ ID NO:15.

### BRIEF DESCRIPTION OF THE DRAWINGS

FIG. 1 illustrates dose escalation study design of anti-ILT4 antibody MAB1.

FIGS. 2A and 2B show serum concentrations of MAB1 following intravenous doses from 3 mg to 1600 mg MAB1 in cycle 1, with Y-axis in log scale (A) or linear scale (B).

FIG. 3 shows percentage of membrane receptor occupancy following intravenous doses from 3 mg to 1600 mg MAB1 in cycle 1.

FIG. 4 is a waterfall plot demonstrating efficacy of MAB1 monotherapy or in combination therapy with pembrolizumab.

FIG. 5 shows percentage of tumor size change over time by different dosages of MAB1.

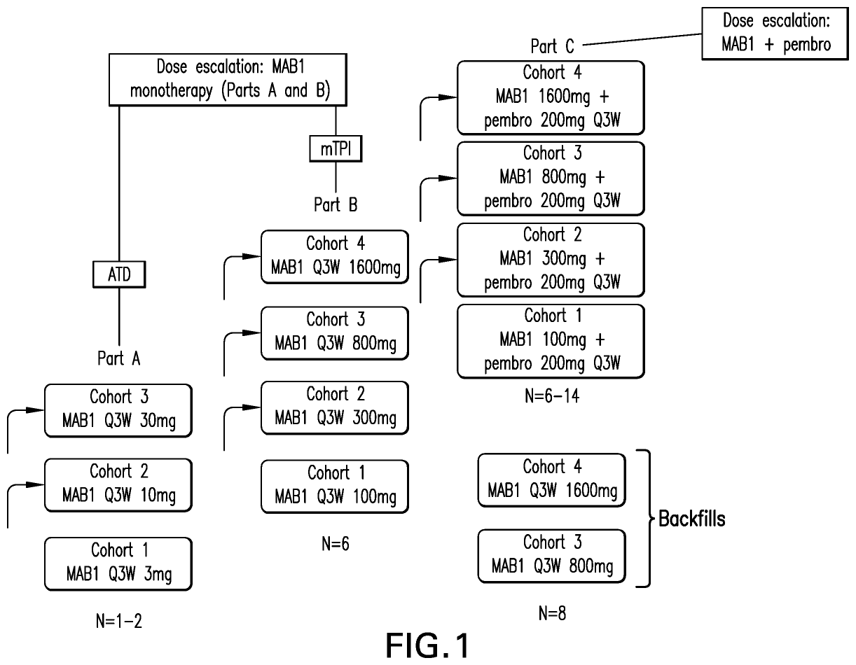

### DETAILED DESCRIPTION

Abbreviations. Throughout the detailed description and examples of the invention the following abbreviations will be used:
BOR Best overall response
CDR Complementarity determining region
CR Complete Response
DFS Disease free survival
DLT Dose limiting toxicity
FR Framework region
IgG Immunoglobulin G
irRC Immune related response criteria
IV Intravenous
NCBI National Center for Biotechnology Information
NCI National Cancer Institute
OS Overall survival
PD Progressive disease
PD-1 Programmed Death 1
PD-L1 Programmed Cell Death 1 Ligand 1
PD-L2 Programmed Cell Death 1 Ligand 2
PFS Progression free survival
PR Partial response
Q3W One dose every three weeks
RECIST Response Evaluation Criteria in Solid Tumors
SD Stable disease
VH Immunoglobulin heavy chain variable region
VL Immunoglobulin light chain variable region

### I. Definitions

Certain technical and scientific terms are specifically defined below. Unless specifically defined elsewhere in this document, all other technical and scientific terms used herein have the meaning commonly understood by one of ordinary skill in the art to which this disclosure relates. “About” when used to modify a numerically defined parameter (e.g., the dose of an anti-PD-1 antibody or antigen binding fragment thereof, an anti-ILT4 antibody or antigen binding fragment thereof, or the length of treatment time with a combination therapy described herein) means that the parameter is within 20%, within 15%, within 10%, within 9%, within 8%, within 7%, within 6%, within 5%, within 4%, within 3%, within 2%, within 1%, or less of the stated numerical value or range for that parameter; where appropriate, the stated parameter may be rounded to the nearest whole number. For example, a dose of about 5 mg/kg may vary between 4.5 mg/kg and 5.5 mg/kg. When referring to the amount of time between administrations in a therapeutic treatment regimen (i.e., amount of time between administrations of the anti-ILT4 antibody, e.g. “about 3 weeks,” which is used interchangeably herein with “approximately every three weeks”), “about” refers to the stated time±a variation that can occur due to patient/clinician scheduling and availability around the 3-week target date. For example, “about 3 weeks” can refer to 3 weeks±5 days, 3 weeks±4 days, 3 weeks±3 days, 3 weeks±2 days or 3 weeks±1 day, or may refer to 2 weeks, 2 days through 3 weeks, 5 days.

As used herein, including the appended claims, the singular forms of words such as “a,” “an,” and “the,” include their corresponding plural references unless the context clearly dictates otherwise.

The terms “administration” or “administer” refers to the act of injecting or otherwise physically delivering a substance as it exists outside the body (e.g., an anti-PD-1 antibody, an anti-ILT4 antibody, as described herein) into a patient, such as by oral, mucosal, intradermal, intravenous, intramuscular delivery, and/or any other methods of physical delivery described herein or known in the art.

“PD-1 antagonist” means any chemical compound or biological molecule that blocks binding of PD-L1 to PD-1 and preferably also blocks binding of PD-L2 to PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, medicaments and disclosed uses in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

As used herein, the term “antibody” refers to any form of immunoglobulin molecule that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic. As used herein, the term “antibody” encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

“Variable region” or “variable domain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “V_{H}.” The variable region of the light chain may be referred to as “V_{L}.” Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody V_{H} β-sheet framework, or one of three hypervariable regions (L1, L2, or L3) within the non-framework region of the antibody V_{L} β-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved b-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

TABLE 1
Correspondence between the CDR Numbering Systems

| | Kabat + | | | | | |
|---|---|---|---|---|---|---|
| | Chothia | IMGT | Kabat | AbM | Chothia | Contact |
| V_{H} CDR1 | 26-35 | 27-38 | 31-35 | 26-35 | 26-32 | 30-35 |
| V_{H} CDR2 | 50-65 | 56-65 | 50-65 | 50-58 | 52-56 | 47-58 |
| V_{H} CDR3 | 95-102 | 105-117 | 95-102 | 95-102 | 95-102 | 93-101 |
| V_{L} CDR1 | 24-34 | 27-38 | 24-34 | 24-34 | 24-34 | 30-36 |
| V_{L} CDR2 | 50-56 | 56-65 | 50-56 | 50-56 | 50-56 | 46-55 |
| V_{L} CDR3 | 89-97 | 105-117 | 89-97 | 89-97 | 89-97 | 89-96 |

“Chimeric antibody” refers to an antibody in which a portion of the heavy and/or light chain contains sequences derived from a particular species (e.g., human) or belonging to a particular antibody class or subclass, while the remainder of the chain(s) is derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences or derivatives thereof. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences or derivatives thereof, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” may be added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, unless otherwise indicated, “antibody fragment” or “antigen binding fragment” refers to a fragment of an antibody that retains the ability to bind specifically to the antigen, e.g., fragments that retain one or more CDR regions. An antibody that “specifically binds to” PD-1 or ILT4 is an antibody that exhibits preferential binding to PD-1 or ILT4 (as appropriate) as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, will bind to the target protein with an affinity that is at least two-fold greater, preferably at least ten times greater, more preferably at least 20-times greater, and most preferably at least 100-times greater than the affinity with non-target proteins.

Antigen binding portions include, for example, Fab, Fab′, F(ab′)2, Fd, Fv, fragments including CDRs, and single chain variable fragment antibodies (scFv), and polypeptides that contain at least a portion of an immunoglobulin that is sufficient to confer specific antigen binding to the antigen (e.g., PD-1 or ILT4). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

When used in connection with selecting from a list, the terms “at least one” item or “one or more” item each include a single item selected from the list as well as mixtures of two or more items selected from the list.

As used herein, the term “immune response” relates to any one or more of the following: specific immune response, non-specific immune response, both specific and non-specific response, innate response, primary immune response, adaptive immunity, secondary immune response, memory immune response, immune cell activation, immune cell-proliferation, immune cell differentiation, and cytokine expression.

The term “patient” (alternatively “subject”) as used herein refers to a mammal that has been the object of treatment, observation, or experiment. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), lagomorphs (e.g., rabbits), rodents (e.g., rats or mice), Procyon lotor (e.g., raccoons). In particular embodiments, the subject is human.

“Biotherapeutic agent” means a cell (such as a CAR-T cell), a vaccine (such as an anti-tumor vaccine), a biological molecule (such as an antibody, antibody-drug conjugate, fusion protein, peptide, nucleic acid, etc.), that enhances anti-tumor immune response and/or suppresses tumor growth.

“Chemotherapeutic agent” refers to a chemical substance that can cause death of cancer cells, or interfere with growth, division, repair, and/or function of cancer cells. Classes of chemotherapeutic agents include but are not limited to alkylating agents, antimetabolites, plant alkaloids, antitumor antibiotics, topoisomerase inhibitors, etc.

The therapeutic agents and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The therapeutic agents and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific therapeutic agent being used, the rate of absorption desired, specific formulation or dosage form used, type or severity of the disorder being treated, the specific site of action, and conditions of the patient, and can be readily selected by a person skilled in the art.

The term “variant” when used in relation to an antibody (e.g., an anti-PD-1 antibody or an anti-ILT4 antibody) or an amino acid region within the antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a variant of an anti-PD-1 antibody may result from one or more (such as, for example, about 1 to about 25, about 1 to about 20, about 1 to about 15, about 1 to about 10, or about 1 to about 5) changes to an amino acid sequence of a native or previously unmodified anti-PD-1 antibody. Variants may be naturally occurring or may be artificially constructed. Polypeptide variants may be prepared from the corresponding nucleic acid molecules encoding the variants. In specific embodiments, an antibody variant (e.g., an anti-PD-1 antibody variant or an anti-ILT4 antibody variant) at least retains the antibody functional activity. In specific embodiments, an anti-PD-1 antibody variant binds to PD-1 and/or is antagonistic to PD-1 activity. In some embodiments, an anti-ILT4 antibody variant binds to ILT4 and/or is antagonistic to ILT4 activity.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2 below.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared ×100. For example, if 8 of 10 of the positions in two sequences are matched when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

“RECIST 1.1 Response Criteria” as used herein means the definitions set forth in Eisenhauer, E. A. et al., Eur. J. Cancer 45:228-247 (2009) for target lesions or nontarget lesions, as appropriate based on the context in which response is being measured.

“Treat” or “treating” cancer as used herein means to administer at least one therapeutic agent to a subject having cancer or diagnosed with cancer to achieve at least one positive therapeutic effect, such as, for example, reduced number of cancer cells, reduced tumor size, reduced rate of cancer cell infiltration into peripheral organs, or reduced rate of tumor metastasis or tumor growth. Such “treatment” may result in a slowing, interrupting, arresting, controlling, or stopping of the progression of cancer as described herein but does not necessarily indicate a total elimination of the cancer or the symptoms of the cancer. Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C≤10% is considered a high anti-tumor activity level, with T/C (%)=Median tumor volume of the treated/Median tumor volume of the control ×100. In some embodiments, the treatment achieved by a combination therapy of the disclosure is any of PR, CR, OR, PFS, DFS, and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some embodiments, response to a combination therapy of the disclosure is any of PR, CR, PFS, DFS, or that is assessed using RECIST 1.1 response criteria. The treatment regimen for a combination therapy of the disclosure that is effective to treat a cancer patient may vary according to factors such as the disease state, age, and weight of the patient, and the ability of the therapy to elicit an anti-cancer response in the subject. While an embodiment of any of the aspects of the disclosure may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

As used herein, the terms “combination,” “combination therapy,” and “therapeutic combination” refer to treatments in which an anti-human PD-1 monoclonal antibody or antigen-binding fragment thereof, an anti-human ILT4 monoclonal antibody or antigen-binding fragment thereof, and optionally additional therapeutic agents, each are administered to a patient in a coordinated manner, over an overlapping period of time. The period of treatment with the anti-human PD-1 monoclonal antibody (or antigen-binding fragment thereof) (the “anti-PD-1 treatment”) is the period of time that a patient undergoes treatment with the anti-human PD-1 monoclonal antibody (or antigen-binding fragment thereof); that is, the period of time from the initial dosing with the anti-human PD-1 monoclonal antibody (or antigen-binding fragment thereof) through the final day of a treatment cycle. Similarly, the period of treatment with the anti-human ILT4 monoclonal antibody (or antigen-binding fragment thereof) (the “anti-ILT4 treatment”) is the period of time that a patient undergoes treatment with the anti-human ILT4 monoclonal antibody (or antigen-binding fragment thereof); that is, the period of time from the initial dosing with the anti-human ILT4 monoclonal antibody (or antigen-binding fragment thereof) through the final day of a treatment cycle. In the methods and therapeutic combinations described herein, the anti-PD-1 treatment overlaps by at least one day with the anti-ILT4 treatment. In certain embodiments, the anti-PD-1 treatment and the anti-ILT4 treatment are the same period of time. In some embodiments, the anti-PD-1 treatment begins prior to the anti-ILT4 treatment. In other embodiments, the anti-PD-1 treatment begins after the anti-ILT4 treatment. In certain embodiments, the anti-PD-1 treatment is terminated prior to termination of the anti-ILT4 treatment. In other embodiments, the anti-PD-1 treatment is terminated after termination of the anti-ILT4 treatment.

The terms “treatment regimen,” “dosing protocol,” and “dosing regimen” are used interchangeably to refer to the dose and timing of administration of a therapeutic agent in a monotherapy or the dose and timing of administration of each therapeutic agent in a combination therapy of the disclosure.

“Tumor” as it applies to a subject diagnosed with, or suspected of having, a cancer refers to a malignant or potentially malignant neoplasm or tissue mass of any size, and includes primary tumors and secondary neoplasms. Non-limiting examples of tumors include solid tumor (e.g., sarcoma (such as chondrosarcoma), carcinoma (such as colon carcinoma), blastoma (such as hepatoblastoma), etc.) and blood tumor (e.g., leukemia (such as acute myeloid leukemia (AML)), lymphoma (such as DLBCL), multiple myeloma (MM), etc.).

The term “tumor volume” or “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CT or MRI scans.

“Co-administration” or “co-administer” as used herein for agents (such as a PD-1 antagonist or anti-ILT4 antibody) means that the agents are administered so as to have overlapping therapeutic activities, and not necessarily that the agents are administered simultaneously to the subject. The agents can be administered concurrently or sequentially. The agents may or may not be in physical combination prior to administration. In an embodiment, the agents are administered to a subject simultaneously or at about the same time. For example, an anti-PD-1 antibody and an anti-ILT4 antibody are contained in separate vials, when in liquid solution, and may be mixed into the same intravenous infusion bag or injection device, and administered simultaneously to the patient. In another embodiment, the agents are administered to a subject sequentially or one after another. For example, an anti-PD-1 antibody and an anti-ILT4 antibody are contained in separate vials, when in liquid solution, and are administered sequentially to the patient. In one embodiment, the anti-PD-1 antibody is administered before the anti-ILT4 antibody. In another embodiment, the anti-PD-1 antibody is administered after the anti-ILT4 antibody.

“Co-formulate” or “co-formulation” as used herein refers to at least two different agents (e.g., a PD-1 antagonist and an anti-ILT4 antibody) that are formulated in one pharmaceutical composition and stored in a single vial or vessel (for example, an injection device) rather than being formulated in separate pharmaceutical compositions and stored individually. In one embodiment, the co-formulation contains two different agents. In a specific embodiment, the co-formulation comprises two different antibodies or antigen binding fragments thereof.

“Anti-tumor response” when referring to a cancer patient treated with a therapeutic regimen, such as a combination therapy described herein, means at least one positive therapeutic effect, such as, reduced number of cancer cells, reduced tumor size, reduced rate of cancer cell infiltration into peripheral organs, reduced rate of tumor metastasis or tumor growth, or progression free survival. Positive therapeutic effects in cancer can be measured in a number of ways (See, W. A. Weber, J. Null. Med. 50:1S-10S (2009); Eisenhauer et al., supra). In some embodiments, an anti-tumor response to a combination therapy described herein is assessed using RECIST 1.1 criteria, bidimentional irRC or unidimensional irRC. In some embodiments, an anti-tumor response is any of SD, PR, CR, PFS, or DFS.

“Bidimensional irRC” refers to the set of criteria described in Wolchok J D, et al. Guidelines for the evaluation of immune therapy activity in solid tumors: immune-related response criteria. Clin Cancer Res. 2009; 15(23):7412-7420. These criteria utilize bidimensional tumor measurements of target lesions, which are obtained by multiplying the longest diameter and the longest perpendicular diameter (cm²) of each lesion.

“Unidimensional irRC” refers to the set of criteria described in Nishino M, Giobbie-Hurder A, Gargano M, Suda M, Ramaiya N H, Hodi F S. Developing a Common Language for Tumor Response to Immunotherapy: Immune-related Response Criteria using Unidimensional measurements. Clin Cancer Res. 2013; 19(14):3936-3943). These criteria utilize the longest diameter (cm) of each lesion.

“Anti-ILT4 antibody” means an antibody that specifically binds to an ILT4 polypeptide, an ILT4 polypeptide fragment, an ILT4 peptide, or an ILT4 epitope and blocks the interaction between ILT4 and its ligand, for example, HLA-G, HLA-A, HLA-B, HLA-F, and/or ANGPTL (such as ANGPTL1, ANGPTL4, or ANGPTL7).

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. All ranges also are intended to include all included sub-ranges, although not necessarily explicitly set forth. For example, a range of 3 to 7 days is intended to include 3, 4, 5, 6, and 7 days. In addition, the term “or,” as used herein, denotes alternatives that may, where appropriate, be combined; that is, the term “or” includes each listed alternative separately as well as their combination.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the claims.

Unless otherwise defined, all technical and scientific terms used herein have the same meaning as commonly understood by one of ordinary skill in the art to which this disclosure relates. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present disclosure. The materials, methods, and examples are illustrative only and not intended to be limiting.

### II. Anti-ILT4 Antibodies

Any antibodies that bind to an ILT4 polypeptide, an ILT4 polypeptide fragment, an ILT4 peptide, or an ILT4 epitope and block the interaction between ILT4 and HLA-G, HLA-A, HLA-B, HLA-F, and/or ANGPTL (such as ANGPTL1, ANGPTL4, or ANGPTL7) can be used in various methods, pharmaceutical compositions, kits, or uses disclosed herein.

In some embodiments, the anti-ILT4 antibody is an anti-human ILT4 antibody. In certain embodiments, the anti-ILT4 antibody is a monoclonal antibody. In other embodiments, the anti-ILT4 antibody is an anti-human ILT4 monoclonal antibody.

In certain embodiments, the anti-human ILT4 monoclonal antibody or antigen binding fragment thereof comprises a V_{L} CDR1, a V_{L} CDR2, and a V_{L} CDR3 comprising amino acid sequences as set forth in SEQ ID NOS:11, 21, and 13, respectively, and a V_{H} CDR1, a V_{H} CDR2, and a V_{H} CDR3 comprising amino acid sequences as set forth in SEQ ID NOS:16, 22, and 18, respectively.

In some embodiments, the anti-human ILT4 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively.

In other embodiments, the anti-human ILT4 monoclonal antibody or antigen binding fragment thereof comprises a V_{L} region comprising an amino acid sequence as set forth in SEQ ID NO:14, and a V_{H} region comprising an amino acid sequence as set forth in SEQ ID NO:19.

In yet other embodiments, the anti-human ILT4 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10.

In some embodiments, the anti-human ILT4 monoclonal antibody can be any antibody, antigen binding fragment thereof, or variant thereof disclosed in WO 2018/187518 and WO 2019/126514, the disclosures of which are incorporated by reference herein in their entireties.

In various embodiments, the anti-human ILT4 monoclonal antibody or antigen binding fragment thereof comprises a variant of the amino acid sequences of the anti-ILT4 antibodies disclosed herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-human ILT4 monoclonal antibody or antigen binding fragment thereof has a V_{L} domain with at least 95%, 90%, 85%, 80%, 75% or 50% sequence homology to one of the V_{L} domains of the anti-ILT4 antibodies described herein, and exhibits specific binding to ILT4. In another embodiment, the anti-human ILT4 monoclonal antibody or antigen binding fragment thereof has a V_{H} domain with at least 95%, 90%, 85%, 80%, 75% or 50% sequence homology to one of the V_{H} domains of the anti-ILT4 antibodies described herein, and exhibits specific binding to ILT4. In yet another embodiment, the anti-human ILT4 monoclonal antibody or antigen binding fragment thereof has a V_{L} domain with at least 95%, 90%, 85%, 80%, 75% or 50% sequence homology to one of the V_{L} domains of the anti-ILT4 antibodies described herein and a V_{H} domain with at least 95%, 90%, 85%, 80%, 75% or 50% sequence homology to one of the V_{H} domains of the anti-ILT4 antibodies described herein, and exhibits specific binding to ILT4.

In one embodiment, the anti-human ILT4 monoclonal antibody or antigen binding fragment thereof has a V_{L} domain having up to 1, 2, 3, 4, 5 or more amino acid substitutions, deletions and/or additions in one of the V_{L} domains of the anti-ILT4 antibodies described herein, and exhibits specific binding to ILT4. In another embodiment, the anti-human ILT4 monoclonal antibody or antigen binding fragment thereof has a V_{H} domain having up to 1, 2, 3, 4, 5 or more amino acid substitutions, deletions, and/or additions in one of the V_{H} domains of the anti-ILT4 antibodies described herein, and exhibits specific binding to ILT4. In yet another embodiment, the anti-human ILT4 monoclonal antibody or antigen binding fragment thereof has a V_{L} domain having up to 1, 2, 3, 4, 5 or more amino acid substitutions, deletions, and/or additions in one of the V_{L} domains of the anti-ILT4 antibodies described herein and a V_{H} domain having up to 1, 2, 3, 4, 5 or more amino acid substitutions, deletions, and/or additions in one of the V_{H} domains of the anti-ILT4 antibodies described herein, and exhibits specific binding to ILT4.

In various embodiments, the anti-human ILT4 monoclonal antibody or antigen binding fragment thereof is selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG₄ constant domain, for example, may be used. In various embodiments, the heavy chain constant domain contains one or more amino acid mutations (e.g., IgG₄ with S228P mutation) to generate desired characteristics of the antibody. These desired characteristics include but are not limited to modified effector functions, physical or chemical stability, half-life of antibody, etc.

Ordinarily, amino acid sequence variants of the anti-ILT4 monoclonal antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

In some embodiments, the anti-human ILT4 monoclonal antibody is a human antibody. In other embodiments, the anti-human ILT4 monoclonal antibody is a humanized antibody.

In some embodiments, the light chain of the anti-human ILT4 monoclonal antibody has a human kappa backbone. In other embodiments, the light chain of the anti-human ILT4 monoclonal antibody has a human lambda backbone.

In some embodiments, the heavy chain of the anti-human ILT4 monoclonal antibody has a human IgG1 backbone. In other embodiments, the heavy chain of the anti-human ILT4 monoclonal antibody has a human IgG2 backbone. In yet other embodiments, the heavy chain of the anti-human ILT4 monoclonal antibody has a human IgG3 backbone. In still other embodiments, the heavy chain of the anti-human ILT4 monoclonal antibody has a human IgG4 backbone.

In some embodiments, the heavy chain of the anti-human ILT4 monoclonal antibody has a human IgG1 variant backbone. In other embodiments, the heavy chain of the anti-human ILT4 monoclonal antibody has a human IgG2 variant backbone. In yet other embodiments, the heavy chain of the anti-human ILT4 monoclonal antibody has a human IgG3 variant backbone. In still other embodiments, the heavy chain of the anti-human ILT4 monoclonal antibody has a human IgG4 variant (e.g., IgG4 with S228P mutation) backbone.

### III. PD-1 Antagonists

Provided herein are PD-1 antagonists that can be used in the various methods, pharmaceutical compositions, kits, and uses disclosed herein, including any chemical compound or biological molecule that blocks binding of PD-L1 to PD-1 and preferably also blocks binding of PD-L2 to PD-1.

In certain embodiments, the PD-1 antagonist is an anti-PD-1 antibody. In other embodiments, the PD-1 antagonist is an anti-PD-L1 antibody.

In some embodiments, the anti-PD-1 antibody is an anti-human PD-1 antibody. In certain embodiments, the anti-PD-1 antibody is a monoclonal antibody. In other embodiments, the anti-PD-1 antibody is an anti-human PD-1 monoclonal antibody.

In some embodiments, the anti-PD-L1 antibody is an anti-human PD-L1 antibody. In certain embodiments, the anti-PD-L1 antibody is a monoclonal antibody. In other embodiments, the anti-PD-1 antibody is an anti-human PD-L1 monoclonal antibody.

Any monoclonal antibodies that bind to a PD-1 polypeptide, a PD-1 polypeptide fragment, a PD-1 peptide, or a PD-1 epitope and block the interaction between PD-1 and its ligand PD-L1 or PD-L2 can be used. In some embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1. In other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L2. In yet other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1 and the interaction between PD-1 and PD-L2.

Any monoclonal antibodies that bind to a PD-L1 polypeptide, a PD-L1 polypeptide fragment, a PD-L1 peptide, or a PD-L1 epitope and block the interaction between PD-L1 and PD-1 can also be used.

In certain embodiments, the anti-human PD-1 monoclonal antibody is selected from the group consisting of pembrolizumab, nivolumab, cemiplimab, pidilizumab (U.S. Pat. No. 7,332,582), AMP-514 (MedImmune LLC, Gaithersburg, MD), PDR001 (U.S. Pat. No. 9,683,048), BGB-A317 (U.S. Pat. No. 8,735,553), MGA012 (MacroGenics, Rockville, MD), sintilimab (Innovent Biologics, Inc., China), tislelizumab (BeiGene, China), camrelizumab (Jiangsu Hengrui Medicine, China), and toripalimab (Junshi Biosciences, China). In one embodiment, the anti-human PD-1 monoclonal antibody is pembrolizumab. In another embodiment, the anti-human PD-1 monoclonal antibody is nivolumab. In another embodiment, the anti-human PD-1 monoclonal antibody is cemiplimab. In yet another embodiment, the anti-human PD-1 monoclonal antibody is pidilizumab. In one embodiment, the anti-human PD-1 monoclonal antibody is AMP-514. In another embodiment, the anti-human PD-1 monoclonal antibody is PDR001. In yet another embodiment, the anti-human PD-1 monoclonal antibody is BGB-A317. In still another embodiment, the anti-human PD-1 monoclonal antibody is MGA012. In one embodiment, the anti-human PD-1 monoclonal antibody is sintilimab. In another embodiment, the anti-human PD-1 monoclonal antibody is tislelizumab. In yet another embodiment, the anti-human PD-1 monoclonal antibody is camrelizumab. In still another embodiment, the anti-human PD-1 monoclonal antibody is toripalimab.

In some embodiments, the anti-human PD-1 monoclonal antibody can be any antibody, antigen binding fragment thereof, or variant thereof disclosed in U.S. Pat. Nos. 7,488,802, 7,521,051, 8,008,449, 8,354,509, 8,168,757, WO2004/004771, WO2004/072286, WO2004/056875, US2011/0271358, and WO 2008/156712, the disclosures of which are incorporated by reference herein in their entireties.

Examples of monoclonal antibodies that bind to human PD-L1 that can be used in various methods, pharmaceutical compositions, kits, and uses described herein are disclosed in WO2013/019906, WO2010/077634, and U.S. Pat. No. 8,383,796, the disclosures of which are incorporated by reference herein in their entireties. Specific anti-human PD-L1 monoclonal antibodies useful as the PD-1 antagonist in the various methods, pharmaceutical compositions, kits, and uses described include atezolizumab, durvalumab, avelumab, BMS-936559, and an antibody comprising the heavy chain and light chain variable regions of SEQ ID NO:20 and SEQ ID NO:21, respectively, of WO2013/019906.

Other PD-1 antagonists useful in various methods, pharmaceutical compositions, kits, and uses described herein include an immunoadhesion molecule that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342, the disclosures of which are incorporated by reference herein in their entireties. Specific fusion proteins useful as the PD-1 antagonist in various methods, kits, and uses described herein include AMP-224 (also known as B7-DCIg), which is a PD-L2-Fc fusion protein and binds to human PD-1.

In various embodiments, the anti-human PD-1 or anti-human PD-L1 monoclonal antibody or antigen binding fragment thereof comprises a variant of the amino acid sequences of the anti-human PD-1 or anti-human PD-L1 antibodies described herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-human PD-1 or anti-human PD-L1 monoclonal antibody or antigen binding fragment thereof has a V_{L} domain with at least 95%, 90%, 85%, 80%, 75% or 50% sequence homology to one of the V_{L} domains of the anti-human PD-1 or anti-human PD-L1 antibodies described herein, and exhibits specific binding to PD-1 or PD-L1. In another embodiment, the anti-human PD-1 or anti-human PD-L1 monoclonal antibody or antigen binding fragment thereof has a V_{H} domain with at least 95%, 90%, 85%, 80%, 75% or 50% sequence homology to one of the V_{H} domains of the anti-human PD-1 or anti-human PD-L1 antibodies described herein, and exhibits specific binding to PD-1 or PD-L1. In yet another embodiment, the anti-human PD-1 or anti-human PD-L1 monoclonal antibody or antigen binding fragment thereof has a V_{L} domain with at least 95%, 90%, 85%, 80%, 75% or 50% sequence homology to one of the V_{L} domains of the anti-human PD-1 or anti-human PD-L1 antibodies described herein and a V_{H} domain with at least 95%, 90%, 85%, 80%, 75% or 50% sequence homology to one of the V_{H} domains of the anti-human PD-1 or anti-human PD-L1 antibodies described herein, and exhibits specific binding to PD-1 or PD-L1.

In one embodiment, the anti-human PD-1 or anti-human PD-L1 monoclonal antibody or antigen binding fragment thereof has a V_{L} domain having up to 1, 2, 3, 4, 5 or more amino acid substitutions, deletions and/or additions in one of the V_{L} domains of the anti-human PD-1 or anti-human PD-L1 antibodies described herein, and exhibits specific binding to PD-1 or PD-L1. In another embodiment, the anti-human PD-1 or anti-human PD-L1 monoclonal antibody or antigen binding fragment thereof has a V_{H} domain having up to 1, 2, 3, 4, 5 or more amino acid substitutions, deletions, and/or additions in one of the V_{H} domains of the anti-human PD-1 or anti-human PD-L1 antibodies described herein, and exhibits specific binding to PD-1 or PD-L1. In yet another embodiment, the anti-human PD-1 or anti-human PD-L1 monoclonal antibody or antigen binding fragment thereof has a V_{L} domain having up to 1, 2, 3, 4, 5 or more amino acid substitutions, deletions, and/or additions in one of the V_{L} domains of the anti-human PD-1 or anti-human PD-L1 antibodies described herein and a V_{H} domain having up to 1, 2, 3, 4, 5 or more amino acid substitutions, deletions, and/or additions in one of the V_{H} domains of the anti-human PD-1 or anti-human PD-L1 antibodies described herein, and exhibits specific binding to PD-1 or PD-L1.

In various embodiments, the anti-human PD-1 or anti-human PD-L1 monoclonal antibody or antigen binding fragment thereof is selected from any class of immunoglobulins, including IgM, IgG, IgD, IgA, and IgE. Preferably, the antibody is an IgG antibody. Any isotype of IgG can be used, including IgG1, IgG2, IgG3, and IgG4. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-human PD-1 or anti-human PD-L1 monoclonal antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

In some embodiments, the anti-human PD-1 or anti-human PD-L1 monoclonal antibody is a human antibody. In other embodiments, the anti-human PD-1 or anti-human PD-L1 monoclonal antibody is a humanized antibody.

In some embodiments, the light chain of the anti-human PD-1 or anti-human PD-L1 monoclonal antibody has a human kappa backbone. In other embodiments, the light chain of the anti-human PD-1 or anti-human PD-L1 monoclonal antibody has a human lambda backbone.

In some embodiments, the heavy chain of the anti-human PD-1 or anti-human PD-L1 monoclonal antibody has a human IgG1 backbone. In other embodiments, the heavy chain of the anti-human PD-1 or anti-human PD-L1 monoclonal antibody has a human IgG2 backbone. In yet other embodiments, the heavy chain of the anti-human PD-1 or anti-human PD-L1 monoclonal antibody has a human IgG3 backbone. In still other embodiments, the heavy chain of the anti-human PD-1 or anti-human PD-L1 monoclonal antibody has a human IgG4 backbone.

In some embodiments, the heavy chain of the anti-human PD-1 or anti-human PD-L1 monoclonal antibody has a human IgG1 variant backbone. In other embodiments, the heavy chain of the anti-human PD-1 or anti-human PD-L1 monoclonal antibody has a human IgG2 variant backbone. In yet other embodiments, the heavy chain of the anti-human PD-1 or anti-human PD-L1 monoclonal antibody has a human IgG3 variant backbone. In still other embodiments, the heavy chain of the anti-human PD-1 or anti-human PD-L1 monoclonal antibody has a human IgG4 variant (e.g., IgG4 with S228P mutation) backbone.

### IV. Methods and Uses

Provided herein are methods of treating cancer in a patient comprising administering to the patient a certain dosage of an anti-ILT4 antibody, either alone or in combination with a certain dosage of another agent (e.g., a PD-1 antagonist, such as an anti-PD-1 antibody or an anti-PD-L1 antibody).

In one aspect, provided herein is a method for treating cancer in a patient comprising administering to the patient 3-1600 mg of an anti-ILT4 antibody, wherein the anti-ILT4 antibody is an anti-ILT4 antibody disclosed in Section II.

In some embodiments, provided herein is a method for treating cancer in a patient comprising administering to the patient 3-1600 mg of an anti-ILT4 antibody, wherein the anti-ILT4 antibody comprises: (a) a light chain variable domain comprising CDR-L1 of SEQ ID NO:11, CDR-L2 of SEQ ID NO:21, and CDR-L3 of SEQ ID NO:13; and (b) a heavy chain variable domain comprising CDR-H1 of SEQ ID NO:16, CDR-H2 of SEQ ID NO:22, and CDR-H3 of SEQ ID NO:18.

In certain embodiments, provided herein is a method for treating cancer in a patient comprising administering to the patient 3-1600 mg of an anti-ILT4 antibody, wherein the anti-ILT4 antibody comprises: (a) a light chain variable domain comprising CDR-L1 of SEQ ID NO:11, CDR-L2 of SEQ ID NO:12, and CDR-L3 of SEQ ID NO:13; and (b) a heavy chain variable domain comprising CDR-H1 of SEQ ID NO:16, CDR-L2 of SEQ ID NO:17, and CDR-H3 of SEQ ID NO:18.

In other embodiments, provided herein is a method for treating cancer in a patient comprising administering to the patient 3-1600 mg of an anti-ILT4 antibody, wherein the anti-ILT4 antibody or antigen binding fragment thereof comprises a heavy chain variable region of SEQ ID NO:19 and a light chain variable region of SEQ ID NO:14.

In yet other embodiments, provided herein is a method for treating cancer in a patient comprising administering to the patient 3-1600 mg of an anti-ILT4 antibody, wherein the anti-ILT4 antibody or antigen binding fragment thereof comprises a heavy chain of SEQ ID NO:20 and a light chain of SEQ ID NO:15.

In some embodiments, the anti-ILT4 antibody is administered via intravenous infusion. In various embodiments, the patient is administered 10-1600 mg, 20-1600 mg, 30-1600 mg, 100-1600 mg, 200-1600 mg, 300-1600 mg, 10-800 mg, 20-800 mg, 30-800 mg, 100-800 mg, 300-800 mg, 30-300 mg, 100-300 mg, or 800-1600 mg of the anti-ILT4 antibody. In certain embodiments, the patient is administered 300-1600 mg of the anti-ILT4 antibody. In some embodiments, the patient is administered 300-800 mg of the anti-ILT4 antibody. In other embodiments, the patient is administered 100-1600 mg of the anti-ILT4 antibody. In yet other embodiments, the patient is administered 100-800 mg of the anti-ILT4 antibody. In still other embodiments, the patient is administered 800-1600 mg of the anti-ILT4 antibody.

In one embodiment, the patient is administered 3 mg of the anti-ILT4 antibody. In another embodiment, the patient is administered 10 mg of the anti-ILT4 antibody. In yet another embodiment, the patient is administered 30 mg of the anti-ILT4 antibody. In still another embodiment, the patient is administered 100 mg of the anti-ILT4 antibody. In one embodiment, the patient is administered 300 mg of the anti-ILT4 antibody. In another embodiment, the patient is administered 800 mg of the anti-ILT4 antibody. In yet another embodiment, the patient is administered 1600 mg of the anti-ILT4 antibody.

In some embodiments, the patient is administered the anti-ILT4 antibody on Day 1 and then once approximately every three weeks thereafter.

In another aspect, provided herein is a method for treating cancer in a patient comprising administering to the patient 3-1600 mg of an anti-ILT4 antibody and another agent, wherein the anti-ILT4 antibody comprises: (a) a light chain variable domain comprising CDR-L1 of SEQ ID NO:11, CDR-L2 of SEQ ID NO:21, and CDR-L3 of SEQ ID NO:13; and (b) a heavy chain variable domain comprising CDR-H1 of SEQ ID NO:16, CDR-H2 of SEQ ID NO:22, and CDR-H3 of SEQ ID NO:18.

In certain embodiments, provided herein is a method for treating cancer in a patient comprising administering to the patient 3-1600 mg of an anti-ILT4 antibody and another agent, wherein the anti-ILT4 antibody comprises: (a) a light chain variable domain comprising CDR-L1 of SEQ ID NO:11, CDR-L2 of SEQ ID NO:12, and CDR-L3 of SEQ ID NO:13; and (b) a heavy chain variable domain comprising CDR-H1 of SEQ ID NO:16, CDR-L2 of SEQ ID NO:17, and CDR-H3 of SEQ ID NO:18.

In other embodiments, provided herein is a method for treating cancer in a patient comprising administering to the patient 3-1600 mg of an anti-ILT4 antibody and another agent, wherein the anti-ILT4 antibody or antigen binding fragment thereof comprises a heavy chain variable region of SEQ ID NO:19 and a light chain variable region of SEQ ID NO:14.

In yet other embodiments, provided herein is a method for treating cancer in a patient comprising administering to the patient 3-1600 mg of an anti-ILT4 antibody and another agent, wherein the anti-ILT4 antibody or antigen binding fragment thereof comprises a heavy chain of SEQ ID NO:20 and a light chain of SEQ ID NO:15.

In some embodiments, provided herein is a method for treating cancer in a patient comprising administering to the patient 3-1600 mg of an anti-ILT4 antibody and a PD-1 antagonist, wherein the anti-ILT4 antibody comprises: (a) a light chain variable domain comprising CDR-L1 of SEQ ID NO:11, CDR-L2 of SEQ ID NO:21, and CDR-L3 of SEQ ID NO:13; and (b) a heavy chain variable domain comprising CDR-H1 of SEQ ID NO:16, CDR-H2 of SEQ ID NO:22, and CDR-H3 of SEQ ID NO:18.

In certain embodiments, provided herein is a method for treating cancer in a patient comprising administering to the patient 3-1600 mg of an anti-ILT4 antibody and a PD-1 antagonist, wherein the anti-ILT4 antibody comprises: (a) a light chain variable domain comprising CDR-L1 of SEQ ID NO:11, CDR-L2 of SEQ ID NO:12, and CDR-L3 of SEQ ID NO:13; and (b) a heavy chain variable domain comprising CDR-H1 of SEQ ID NO:16, CDR-L2 of SEQ ID NO:17, and CDR-H3 of SEQ ID NO:18.

In other embodiments, provided herein is a method for treating cancer in a patient comprising administering to the patient 3-1600 mg of an anti-ILT4 antibody and a PD-1 antagonist, wherein the anti-ILT4 antibody or antigen binding fragment thereof comprises a heavy chain variable region of SEQ ID NO:19 and a light chain variable region of SEQ ID NO:14.

In yet other embodiments, provided herein is a method for treating cancer in a patient comprising administering to the patient 3-1600 mg of an anti-ILT4 antibody and a PD-1 antagonist, wherein the anti-ILT4 antibody or antigen binding fragment thereof comprises a heavy chain of SEQ ID NO:20 and a light chain of SEQ ID NO:15.

In certain embodiments, the PD-1 antagonist is an anti-PD-1 antibody or antigen binding fragment thereof. In some embodiments, the PD-1 antagonist is an anti-PD-1 antibody or antigen binding fragment thereof described in Section III.

In other embodiments, the PD-1 antagonist is an anti-PD-L1 antibody or antigen binding fragment thereof. In yet other embodiments, the PD-1 antagonist is an anti-PD-L1 antibody or antigen binding fragment thereof described in Section III.

In some embodiments, the anti-PD-1 antibody or antigen binding fragment thereof comprises: (a) a light chain variable domain comprising CDR-L1 of SEQ ID NO:1, CDR-L2 of SEQ ID NO:2, and CDR-L3 of SEQ ID NO:3; and (b) a heavy chain variable domain comprising CDR-H1 of SEQ ID NO:6, CDR-L2 of SEQ ID NO:7, and CDR-H3 of SEQ ID NO:8.

In other embodiments, the anti-PD-1 antibody or antigen binding fragment thereof comprises a heavy chain variable region of SEQ ID NO:9 and a light chain variable region of SEQ ID NO:4.

In yet other embodiments, the anti-PD-1 antibody or antigen binding fragment thereof comprises a heavy chain of SEQ ID NO:10 and a light chain of SEQ ID NO:5.

In one embodiment, the anti-PD-1 antibody is pembrolizumab.

In another embodiment, the anti-PD-1 antibody is a pembrolizumab variant.

In yet another embodiment, the anti-PD-1 antibody is nivolumab or variant thereof.

In still another embodiment, the anti-PD-1 antibody is cemiplimab or variant thereof.

In one embodiment, the anti-PD-L1 antibody is atezolizumab or variant thereof.

In another embodiment, the anti-PD-L1 antibody is durvalumab or variant thereof.

In yet another embodiment, the anti-PD-L1 antibody is avelumab or variant thereof.

In certain embodiments of various methods provided herein, 200 mg of the anti-PD-1 antibody is administered to the patient via intravenous infusion on Day 1 and then once approximately every three weeks thereafter.

In other embodiments, 400 mg of the anti-PD-1 antibody is administered to the patient via intravenous infusion on Day 1 and then once approximately every six weeks thereafter.

In certain embodiments, the method of treating cancer in a patient comprises administering to the patient 3-1600 mg of anti-ILT4 antibody and 200-400 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:22, and CDR-H3 of SEQ ID NO:18, and a light chain variable region comprising light chain CDR-L1 of SEQ ID NO:11, CDR-L2 of SEQ ID NO:21, and CDR-L3 of SEQ ID NO:13. In certain embodiments, the method of treating cancer in a patient comprises administering to the patient 3-1600 mg of anti-ILT4 antibody and 200-400 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:22, and CDR-H3 of SEQ ID NO:18, and a light chain variable region comprising light chain CDR-L1 of SEQ ID NO:11, CDR-L2 of SEQ ID NO:21, and CDR-L3 of SEQ ID NO:13, wherein the cancer is selected from the group consisting of: melanoma, non-small cell lung cancer, relapsed or refractory classical Hodgkin lymphoma, primary mediastinal large B-cell lymphoma, head and neck squamous cell cancer, urothelial carcinoma, esophageal cancer, stomach cancer, gastric or gastroesophageal junction adenocarcinoma, esophageal or certain gastroesophageal junction carcinomas, gastric cancer, cervical cancer, PMBCL, MSI-H cancer, colon cancer, rectal cancer, hepatocellular carcinoma, Merkel cell carcinoma, renal cell carcinoma, cutaneous squamous cell carcinoma, and triple-negative breast cancer.

In some embodiments, the method of treating cancer in a patient comprises administering to the patient 3-1600 mg of anti-ILT4 antibody and 200-400 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO: 11, CDR-L2 of SEQ ID NO:12, and CDR-L3 of SEQ ID NO:13. In some embodiments, the method of treating cancer in a patient comprises administering to the patient 3-1600 mg of anti-ILT4 antibody and 200-400 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO: 18, and a light chain variable region comprising light chain CDR-L1 of SEQ ID NO:11, CDR-L2 of SEQ ID NO:12, and CDR-L3 of SEQ ID NO:13, melanoma, non-small cell lung cancer, relapsed or refractory classical Hodgkin lymphoma, primary mediastinal large B-cell lymphoma, head and neck squamous cell cancer, urothelial carcinoma, esophageal cancer, stomach cancer, gastric or gastroesophageal junction adenocarcinoma, esophageal or certain gastroesophageal junction carcinomas, gastric cancer, cervical cancer, PMBCL, MSI-H cancer, colon cancer, rectal cancer, hepatocellular carcinoma, Merkel cell carcinoma, renal cell carcinoma, cutaneous squamous cell carcinoma, and triple-negative breast cancer.

In other embodiments, the method of treating cancer in a patient comprises administering to the patient 3-1600 mg of anti-ILT4 antibody and 200-400 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14. In other embodiments, the method of treating cancer in a patient comprises administering to the patient 3-1600 mg of anti-ILT4 antibody and 200-400 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14, wherein the cancer is selected from the group consisting of: melanoma, non-small cell lung cancer, relapsed or refractory classical Hodgkin lymphoma, primary mediastinal large B-cell lymphoma, head and neck squamous cell cancer, urothelial carcinoma, esophageal cancer, stomach cancer, gastric or gastroesophageal junction adenocarcinoma, esophageal or certain gastroesophageal junction carcinomas, gastric cancer, cervical cancer, PMBCL, MSI-H cancer, colon cancer, rectal cancer, hepatocellular carcinoma, Merkel cell carcinoma, renal cell carcinoma, cutaneous squamous cell carcinoma, and triple-negative breast cancer.

In yet other embodiments, the method of treating cancer in a patient comprises administering to the patient 3-1600 mg of anti-ILT4 antibody and 200-400 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15. In yet other embodiments, the method of treating cancer in a patient comprises administering to the patient 3-1600 mg of anti-ILT4 antibody and 200-400 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15, wherein the cancer is selected from the group consisting of: melanoma, non-small cell lung cancer, relapsed or refractory classical Hodgkin lymphoma, primary mediastinal large B-cell lymphoma, head and neck squamous cell cancer, urothelial carcinoma, esophageal cancer, stomach cancer, gastric or gastroesophageal junction adenocarcinoma, esophageal or certain gastroesophageal junction carcinomas, gastric cancer, cervical cancer, PMBCL, MSI-H cancer, colon cancer, rectal cancer, hepatocellular carcinoma, Merkel cell carcinoma, renal cell carcinoma, cutaneous squamous cell carcinoma, and triple-negative breast cancer.

In certain embodiments, 200 mg of the anti-PD-1 antibody is administered to the patient via intravenous infusion on Day 1 and then once approximately every three weeks thereafter, and 100 mg of the anti-ILT4 antibody is administered to the patient via intravenous infusion on Day 1 and then once approximately every three weeks thereafter.

In some embodiments, 400 mg of the anti-PD-1 antibody is administered to the patient via intravenous infusion on Day 1 and then once every about six weeks thereafter, and 100 mg of the anti-ILT4 antibody is administered to the patient via intravenous infusion on Day 1 and then once approximately every three weeks thereafter.

In certain embodiments, 200 mg of the anti-PD-1 antibody is administered to the patient via intravenous infusion on Day 1 and then once approximately every three weeks thereafter, and 300 mg of the anti-ILT4 antibody is administered to the patient via intravenous infusion on Day 1 and then once approximately every three weeks thereafter.

In some embodiments, 400 mg of the anti-PD-1 antibody is administered to the patient via intravenous infusion on Day 1 and then once every about six weeks thereafter, and 300 mg of the anti-ILT4 antibody is administered to the patient via intravenous infusion on Day 1 and then once approximately every three weeks thereafter. In other embodiments, 200 mg of the anti-PD-1 antibody is administered to the patient via intravenous infusion on Day 1 and then once approximately every three weeks thereafter, and 800 mg of the anti-ILT4 antibody is administered to the patient via intravenous infusion on Day 1 and then once approximately every three weeks thereafter.

In yet other embodiments, 400 mg of the anti-PD-1 antibody is administered to the patient via intravenous infusion on Day 1 and then once every about six weeks thereafter, and 800 mg of the anti-ILT4 antibody is administered to the patient via intravenous infusion on Day 1 and then once approximately every three weeks thereafter.

In still other embodiments, 200 mg of the anti-PD-1 antibody is administered to the patient via intravenous infusion on Day 1 and then once approximately every three weeks thereafter, and 1600 mg of the anti-ILT4 antibody is administered to the patient via intravenous infusion on Day 1 and then once approximately every three weeks thereafter.

In yet still other embodiments, 400 mg of the anti-PD-1 antibody is administered to the patient via intravenous infusion on Day 1 and then once every about six weeks thereafter, and 1600 mg of the anti-ILT4 antibody is administered to the patient via intravenous infusion on Day 1 and then once approximately every three weeks thereafter.

In some embodiments of various methods disclosed herein, the anti-PD-1 antibody and the anti-ILT4 antibody are formulated in two separate pharmaceutical compositions. In certain embodiments, the anti-ILT4 antibody is co-administered with a PD-1 antagonist. In one embodiment, the anti-PD-1 antibody and the anti-ILT4 antibody are administered concurrently.

In another embodiment, the anti-PD-1 antibody and the anti-ILT4 antibody are administered sequentially. In one embodiment, the anti-PD-1 antibody is administered before the anti-ILT4 antibody. In another embodiment, the anti-PD-1 antibody is administered after the anti-ILT4 antibody.

In one embodiment, 200 mg of anti-PD-1 antibody and 100-1600 mg of anti-ILT4 antibody are co-administered on Day 1 and then once approximately every three weeks thereafter. In another embodiment, 200 mg of anti-PD-1 antibody and 100 mg of anti-ILT4 antibody are co-administered on Day 1 and then once approximately every three weeks thereafter. In yet another embodiment, 200 mg of anti-PD-1 antibody and 300 mg of anti-ILT4 antibody are co-administered on Day 1 and then once approximately every three weeks thereafter. In still another embodiment, 200 mg of anti-PD-1 antibody and 800 mg of anti-ILT4 antibody are co-administered on Day 1 and then once approximately every three weeks thereafter. In yet still another embodiment, 200 mg of anti-PD-1 antibody and 1600 mg of anti-ILT4 antibody are co-administered on Day 1 and then once approximately every three weeks thereafter.

In certain embodiments of various methods disclosed herein, the anti-PD-1 antibody and the anti-ILT4 antibody are co-formulated in one pharmaceutical composition. In one embodiment, 200 mg of anti-PD-1 antibody is co-formulated with 3-1600 mg, 10-1600 mg, 30-1600 mg, 100-1600 mg, 300-1600 mg, 800-1600 mg, 100-300 mg, 100-800 mg, or 300-800 mg of anti-ILT4 antibody. In another embodiment, 200 mg of anti-PD-1 antibody is co-formulated with 3 mg, 10 mg, 30 mg, 100 mg, 300 mg, 800 mg, or 1600 mg of anti-ILT4 antibody. In one embodiment, 200 mg of anti-PD-1 antibody is co-formulated with 100 mg of anti-ILT4 antibody.

In another embodiment, 200 mg of anti-PD-1 antibody is co-formulated with 300 mg of anti-ILT4 antibody. In yet another embodiment, 200 mg of anti-PD-1 antibody is co-formulated with 800 mg of anti-ILT4 antibody. In still another embodiment, 200 mg of anti-PD-1 antibody is co-formulated with 1600 mg of anti-ILT4 antibody.

In another aspect, provided is use of the anti-ILT4 antibody in the manufacture of a medicament for treating cancer in an individual, wherein the anti-ILT4 antibody is co-administered at 3-1600 mg via intravenous infusion with the anti-PD-1 antibody at 200 mg via intravenous infusion. In one embodiment, the invention provides use of the anti-ILT4 antibody in the manufacture of a medicament for treating cancer in an individual, wherein the anti-ILT4 antibody is co-administered at 100 mg via intravenous infusion with the anti-PD-1 antibody at 200 mg via intravenous infusion. In another embodiment, the invention provides use of the anti-ILT4 antibody in the manufacture of a medicament for treating cancer in an individual, wherein the anti-ILT4 antibody is co-administered at 300 mg via intravenous infusion with the anti-PD-1 antibody at 200 mg via intravenous infusion. In yet another embodiment, the invention provides use of the anti-ILT4 antibody in the manufacture of a medicament for treating cancer in an individual, wherein the anti-ILT4 antibody is co-administered at 800 mg via intravenous infusion with the anti-PD-1 antibody at 200 mg via intravenous infusion. In still another embodiment, the invention provides use of the anti-ILT4 antibody in the manufacture of a medicament for treating cancer in an individual, wherein the anti-ILT4 antibody is co-administered at 1600 mg via intravenous infusion with the anti-PD-1 antibody at 200 mg via intravenous infusion.

In yet another embodiment, provided is use of the anti-ILT4 antibody in the manufacture of a medicament for treating cancer in an individual, wherein 3-1600 mg of the anti-ILT4 antibody is co-administered via intravenous infusion with 400 mg of the anti-PD-1 antibody via intravenous infusion. In one embodiment, the invention provides use of the anti-ILT4 antibody in the manufacture of a medicament for treating cancer in an individual, wherein 100 mg of the anti-ILT4 antibody is co-administered via intravenous infusion with 400 mg of the anti-PD-1 antibody via intravenous infusion. In another embodiment, the invention provides use of the anti-ILT4 antibody in the manufacture of a medicament for treating cancer in an individual, wherein 300 mg of the anti-ILT4 antibody is co-administered via intravenous infusion with 400 mg of the anti-PD-1 antibody via intravenous infusion. In yet another embodiment, the invention provides use of the anti-ILT4 antibody in the manufacture of a medicament for treating cancer in an individual, wherein 800 mg of the anti-ILT4 antibody is co-administered via intravenous infusion with 400 mg of the anti-PD-1 antibody via intravenous infusion. In still another embodiment, the invention provides use of the anti-ILT4 antibody in the manufacture of a medicament for treating cancer in an individual, wherein 1600 mg of the anti-ILT4 antibody is co-administered via intravenous infusion with 400 mg of the anti-PD-1 antibody via intravenous infusion.

Further provided herein is use of an anti-ILT4 antibody (e.g., as described in Section II) and a PD-1 antagonist (e.g., as described in Section III) in the manufacture of a medicament for treating cancer in an individual. In certain embodiments, the PD-1 antagonist is an anti-PD-1 antibody. In other embodiments, the PD-1 antagonist is an anti-PD-L1 antibody. In some embodiments, the medicament comprises the anti-ILT4 antibody and the anti-PD-1 antibody in separate pharmaceutical compositions. In other embodiments, the medicament comprises the anti-ILT4 antibody and the anti-PD-1 antibody in one pharmaceutical composition. In some embodiments, the medicament comprises 200-400 mg of anti-PD-1 antibody and 3-1600 mg of anti-ILT4 antibody. In one embodiment, the medicament comprises 200 mg of anti-PD-1 antibody and 100 mg of anti-ILT4 antibody. In another embodiment, the medicament comprises 200 mg of anti-PD-1 antibody and 300 mg of anti-ILT4 antibody. In yet another embodiment, the medicament comprises 200 mg of anti-PD-1 antibody and 800 mg of anti-ILT4 antibody. In still another embodiment, the medicament comprises 200 mg of anti-PD-1 antibody and 1600 mg of anti-ILT4 antibody. In one embodiment, the medicament comprises 400 mg of anti-PD-1 antibody and 100 mg of anti-ILT4 antibody. In another embodiment, the medicament comprises 400 mg of anti-PD-1 antibody and 300 mg of anti-ILT4 antibody. In yet another embodiment, the medicament comprises 400 mg of anti-PD-1 antibody and 800 mg of anti-ILT4 antibody. In still another embodiment, the medicament comprises 400 mg of anti-PD-1 antibody and 1600 mg of anti-ILT4 antibody.

In certain embodiments of various methods, uses, and medicaments described, the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and the anti-ILT4 antibody comprises a heavy chain variable region comprising heavy chain CDR-H1 of SEQ ID NO:16, CDR-H2 of SEQ ID NO:22, and CDR-H3 of SEQ ID NO:18, and a light chain variable region comprising light chain CDR-L1 of SEQ ID NO: 11, CDR-L2 of SEQ ID NO:21, and CDR-L3 of SEQ ID NO:13.

In certain embodiments of various methods, uses, and medicaments described, the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO: 11, CDR-L2 of SEQ ID NO:12, and CDR-L3 of SEQ ID NO:13.

In some embodiments of various methods, uses, and medicaments described, the anti-PD-1 antibody comprises a heavy chain variable region of SEQ ID NO:9 and a light chain variable region of SEQ ID NO: 4, and the anti-ILT4 antibody comprises a heavy chain variable region of SEQ ID NO:19 and a light chain variable region of SEQ ID NO:14.

In other embodiments of various methods, uses, and medicaments described, the anti-PD-1 antibody comprises a heavy chain of SEQ ID NO:10 and a light chain of SEQ ID NO: 5, and the anti-ILT4 antibody comprises a heavy chain of SEQ ID NO:20 and a light chain of SEQ ID NO:15.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., NSCLC), pleural mesothelioma, gastric cancer, colorectal cancer, cervical cancer, synovial sarcoma, head and neck cancer, squamous cell carcinoma, lymphoma (e.g., diffuse large B-cell lymphoma (DLBCL) or non-Hodgkin lymphoma (NHL)), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelofibrosis, soft tissue sarcoma, thyroid cancer, endometrial cancer, and carcinoid cancer.

In some embodiments of the methods, the cancer is selected from the group consisting of: melanoma, lung cancer, head and neck cancer, bladder cancer, breast cancer, gastrointestinal cancer, multiple myeloma, hepatocellular cancer, merkel cell carcinoma, cutaneous squamous cell carcinoma, lymphoma, renal cancer, mesothelioma, ovarian cancer, esophageal cancer, anal cancer, biliary tract cancer, colorectal cancer, endometrial cancer, cervical cancer, thyroid cancer, salivary cancer, prostate cancer (e.g. hormone refractory prostate adenocarcinoma), pancreatic cancer, colon cancer, liver cancer, thyroid cancer, glioblastoma, glioma, and other neoplastic malignancies.

In some embodiments, the cancer is skin cancer. In some embodiments, the skin cancer is melanoma.

In some embodiments, the cancer is lung cancer. In some embodiments, the lung cancer in non-small cell lung cancer. In alternate embodiments, the lung cancer is small-cell lung cancer.

In some embodiments, the cancer is head and neck squamous cell cancer. In some embodiments, the head and neck cancer is nasopharyngeal cancer. In some embodiments, the cancer is thyroid cancer. In other embodiments, the cancer is salivary cancer. In other embodiments, the cancer is squamous cell carcinoma of the head and neck.

In some embodiments, the cancer is classical Hodgkin lymphoma (cHL). In some embodiments, the lymphoma is Hodgkin lymphoma. In some embodiments, the lymphoma is classical Hodgkin lymphoma (cHL).

In other embodiments, the lymphoma is non-Hodgkin lymphoma. In particular embodiments, the lymphoma is primary mediastinal large B-cell lymphoma (PMBCL). In some embodiments, the lymphoma is diffuse large B-cell lymphoma (DLBCL).

In some embodiments, the breast cancer is triple negative breast cancer.

In further embodiments, the breast cancer is ER+/HER2− breast cancer.

In some embodiments, the bladder cancer is urothelial cancer.

In some embodiments, the head and neck cancer is nasopharyngeal cancer. In some embodiments, the cancer is thyroid cancer. In other embodiments, the cancer is salivary cancer.

In other embodiments, the cancer is squamous cell carcinoma of the head and neck.

In some embodiments, the cancer is metastatic colorectal cancer with high levels of microsatellite instability (MSI-H).

In some embodiments, the cancer is a solid tumor with a high level of microsatellite instability (MSI-H).

In certain embodiments, the cancer is metastatic. In some embodiments, the cancer is relapsed. In other embodiments, the cancer is refractory. In yet other embodiments, the cancer is relapsed and refractory.

In some embodiments of various methods disclosed herein, the cancer is head and neck squamous cell cancer (HNSCC), gastric cancer, pancreatic cancer, glioblastoma (GBM), renal cell carcinoma (RCC), or non-small cell lung cancer (NSCLC). In one embodiment, the cancer is HNSCC. In another embodiment, the cancer is gastric cancer. In yet another embodiment, the cancer is pancreatic cancer. In still another embodiment, the cancer is GBM. In another embodiment, the cancer is RCC. In yet still another embodiment, the cancer is NSCLC.

In one embodiment, provided is a method of treating HNSCC in a patient comprising administering to the patient 3-1600 mg of anti-ILT4 antibody and 200-400 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating HNSCC in a patient comprising administering to the patient 100 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating HNSCC in a patient comprising administering to the patient 300 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO: 11, CDR-L2 of SEQ ID NO:12, and CDR-L3 of SEQ ID NO:13.

In one embodiment, provided is a method of treating HNSCC in a patient comprising administering to the patient 800 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating HNSCC in a patient comprising administering to the patient 1600 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO: 11, CDR-L2 of SEQ ID NO:12, and CDR-L3 of SEQ ID NO:13.

In one embodiment, provided is a method of treating gastric cancer in a patient comprising administering to the patient 3-1600 mg of anti-ILT4 antibody and 200-400 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating gastric cancer in a patient comprising administering to the patient 100 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating gastric cancer in a patient comprising administering to the patient 300 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating gastric cancer in a patient comprising administering to the patient 800 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating gastric cancer in a patient comprising administering to the patient 1600 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO: 13.

In one embodiment, provided is a method of treating pancreatic cancer in a patient comprising administering to the patient 3-1600 mg of anti-ILT4 antibody and 200-400 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating pancreatic cancer in a patient comprising administering to the patient 100 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating pancreatic cancer in a patient comprising administering to the patient 300 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating pancreatic cancer in a patient comprising administering to the patient 800 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating pancreatic cancer in a patient comprising administering to the patient 1600 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating GBM in a patient comprising administering to the patient 3-1600 mg of anti-ILT4 antibody and 200-400 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating GBM in a patient comprising administering to the patient 100 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO: 16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating GBM in a patient comprising administering to the patient 300 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating GBM in a patient comprising administering to the patient 800 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO: 11, CDR-L2 of SEQ ID NO:12, and CDR-L3 of SEQ ID NO:13.

In one embodiment, provided is a method of treating GBM in a patient comprising administering to the patient 1600 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO: 11, CDR-L2 of SEQ ID NO:12, and CDR-L3 of SEQ ID NO:13.

In one embodiment, provided is a method of treating RCC in a patient comprising administering to the patient 3-1600 mg of anti-ILT4 antibody and 200-400 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating RCC in a patient comprising administering to the patient 100 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating RCC in a patient comprising administering to the patient 300 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO: 11, CDR-L2 of SEQ ID NO:12, and CDR-L3 of SEQ ID NO:13.

In one embodiment, provided is a method of treating RCC in a patient comprising administering to the patient 800 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating RCC in a patient comprising administering to the patient 1600 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating NSCLC in a patient comprising administering to the patient 3-1600 mg of anti-ILT4 antibody and 200-400 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO: 11, CDR-L2 of SEQ ID NO: 12, and CDR-L3 of SEQ ID NO:13.

In one embodiment, provided is a method of treating NSCLC in a patient comprising administering to the patient 100 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO: 16, CDR-H2 of SEQ ID NO:17, and CDR-H3 of SEQ ID NO: 18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating NSCLC in a patient comprising administering to the patient 300 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO: 1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating NSCLC in a patient comprising administering to the patient 800 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, provided is a method of treating NSCLC in a patient comprising administering to the patient 1600 mg of anti-ILT4 antibody and 200 mg of anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO: 11, CDR-L2 of SEQ ID NO:12, and CDR-L3 of SEQ ID NO:13.

In one embodiment, provided is a method of treating HNSCC in a patient comprising administering to the patient 3-1600 mg of anti-ILT4 antibody and 200-400 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO: 19 and a light chain variable region of SEQ ID NO:14.

In one embodiment, provided is a method of treating HNSCC in a patient comprising administering to the patient 100 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating HNSCC in a patient comprising administering to the patient 300 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating HNSCC in a patient comprising administering to the patient 800 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating HNSCC in a patient comprising administering to the patient 1600 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating gastric cancer in a patient comprising administering to the patient 3-1600 mg of anti-ILT4 antibody and 200-400 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating gastric cancer in a patient comprising administering to the patient 100 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating gastric cancer in a patient comprising administering to the patient 300 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating gastric cancer in a patient comprising administering to the patient 800 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating gastric cancer in a patient comprising administering to the patient 1600 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating pancreatic cancer in a patient comprising administering to the patient 3-1600 mg of anti-ILT4 antibody and 200-400 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating pancreatic cancer in a patient comprising administering to the patient 100 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating pancreatic cancer in a patient comprising administering to the patient 300 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating pancreatic cancer in a patient comprising administering to the patient 800 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating pancreatic cancer in a patient comprising administering to the patient 1600 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating GBM in a patient comprising administering to the patient 3-1600 mg of anti-ILT4 antibody and 200-400 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating GBM in a patient comprising administering to the patient 100 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating GBM in a patient comprising administering to the patient 300 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating GBM in a patient comprising administering to the patient 800 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating GBM in a patient comprising administering to the patient 1600 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating RCC in a patient comprising administering to the patient 3-1600 mg of anti-ILT4 antibody and 200-400 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating RCC in a patient comprising administering to the patient 100 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating RCC in a patient comprising administering to the patient 300 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating RCC in a patient comprising administering to the patient 800 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating RCC in a patient comprising administering to the patient 1600 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating NSCLC in a patient comprising administering to the patient 3-1600 mg of anti-ILT4 antibody and 200-400 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating NSCLC in a patient comprising administering to the patient 100 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating NSCLC in a patient comprising administering to the patient 300 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating NSCLC in a patient comprising administering to the patient 800 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating NSCLC in a patient comprising administering to the patient 1600 mg of anti-ILT4 antibody and 200 mg of anti-PD-1 antibody, wherein the anti-PD-1 antibody comprises a heavy chain variable region of SEQ ID NO:9 and a light chain variable region of SEQ ID NO: 4, and wherein the anti-ILT4 antibody comprises a heavy chain variable region of SEQ ID NO:19 and a light chain variable region of SEQ ID NO:14.

In one embodiment, provided is a method of treating HNSCC in a patient comprising administering to the patient 3-1600 mg of anti-ILT4 antibody and 200-400 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating HNSCC in a patient comprising administering to the patient 100 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating HNSCC in a patient comprising administering to the patient 300 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating HNSCC in a patient comprising administering to the patient 800 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating HNSCC in a patient comprising administering to the patient 1600 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating gastric cancer in a patient comprising administering to the patient 3-1600 mg of anti-ILT4 antibody and 200-400 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating gastric cancer in a patient comprising administering to the patient 100 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating gastric cancer in a patient comprising administering to the patient 300 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating gastric cancer in a patient comprising administering to the patient 800 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating gastric cancer in a patient comprising administering to the patient 1600 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating pancreatic cancer in a patient comprising administering to the patient 3-1600 mg of anti-ILT4 antibody and 200-400 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating pancreatic cancer in a patient comprising administering to the patient 100 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating pancreatic cancer in a patient comprising administering to the patient 300 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating pancreatic cancer in a patient comprising administering to the patient 800 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating pancreatic cancer in a patient comprising administering to the patient 1600 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating GBM in a patient comprising administering to the patient 3-1600 mg of anti-ILT4 antibody and 200-400 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating GBM in a patient comprising administering to the patient 100 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating GBM in a patient comprising administering to the patient 300 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating GBM in a patient comprising administering to the patient 800 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating GBM in a patient comprising administering to the patient 1600 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating RCC in a patient comprising administering to the patient 3-1600 mg of anti-ILT4 antibody and 200-400 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating RCC in a patient comprising administering to the patient 100 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating RCC in a patient comprising administering to the patient 300 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating RCC in a patient comprising administering to the patient 800 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating RCC in a patient comprising administering to the patient 1600 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating NSCLC in a patient comprising administering to the patient 3-1600 mg of anti-ILT4 antibody and 200-400 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating NSCLC in a patient comprising administering to the patient 100 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating NSCLC in a patient comprising administering to the patient 300 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating NSCLC in a patient comprising administering to the patient 800 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

In one embodiment, provided is a method of treating NSCLC in a patient comprising administering to the patient 1600 mg of anti-ILT4 antibody and 200 mg of anti-PD-1 antibody, wherein the anti-PD-1 antibody comprises a heavy chain of SEQ ID NO:10 and a light chain of SEQ ID NO: 5, and wherein the anti-ILT4 antibody comprises a heavy chain of SEQ ID NO:20 and a light chain of SEQ ID NO:15.

The PD-1 antagonists and the anti-ILT4 antibody can be used with additional therapeutic agents in the various methods, uses, and medicaments disclosed herein.

The additional therapeutic agent can be, e.g., a chemotherapeutic or a biotherapeutic agent (including but not limited to antibodies or antigen binding fragments thereof that specifically bind to an antigen selected from the group consisting of: PD-L1, PD-L2, CTLA4, BTLA, TIM3, HVEM, GITR, CD27, ILT2, ILT3, ILT5, SIRPα, NKG2A, NKG2C, NKG2E, TSLP, IL10, VISTA, VEGF, EGFR, Her2/neu, VEGF receptors, other growth factor receptors, CD20, CD28, CD40, CD-40L, CD70, OX-40, 4-1BB, and ICOS).

The additional therapeutic agent can be selected from the group consisting of STING agonists, poly ADP ribose polymerase (PARP) inhibitors, mitogen-activated protein kinase (MEK) inhibitors, cyclin-dependent kinase (CDK) inhibitors, indoleamine 2,3-dioxygenase (IDO) inhibitors, tryptophan 2,3-dioxygenase (TDO) selective inhibitors, anti-viral compounds, antigens, adjuvants, anti-cancer agents, CTLA-4 pathway antagonists, lipids, liposomes, peptides, cytotoxic agents, chemotherapeutic agents, immunomodulatory cell lines, checkpoint inhibitors, vascular endothelial growth factor (VEGF) receptor inhibitors, topoisomerase II inhibitors, smoothen inhibitors, alkylating agents, anti-tumor antibiotics, anti-metabolites, retinoids, and immunomodulatory agents including but not limited to anti-cancer vaccines.

The additional therapeutic agent can be an anti-viral compound, including but not limited to, hepatitis B virus (HBV) inhibitors, hepatitis C virus (HCV) protease inhibitors, HCV polymerase inhibitors, HCV NS4A inhibitors, HCV NS5A inhibitors, HCV NS5b inhibitors, and human immunodeficiency virus (HIV) inhibitors.

The additional therapeutic agent can be a cytotoxic agent, including but not limited to, arsenic trioxide (sold under the tradename TRISENOX®) and asparaginase (also known as L-asparaginase and Erwinia L-asparaginase, sold under the tradenames ELSPAR® and KIDROLASE®).

The additional therapeutic agent can be an chemotherapeutic agent, including but not limited to, abiraterone acetate, altretamine, anhydrovinblastine, auristatin, bexarotene, bicalutamide, BMS 184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-prolyl-1-Lproline-t-butylamide, cachectin, cemadotin, chlorambucil, cyclophosphamide, 3′,4′-didehydro-4′deoxy-8′-norvin-caleukoblastine, dinaciclib, docetaxol, doxetaxel, cyclophosphamide, carmustine, carboplatin, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyurea and taxanes, ifosfamide, liarozole, lonidamine, lomustine, MDV3100, mechlorethamine (nitrogen mustard), melphalan, mivobulin isethionate, rhizoxin, sertenef, streptozocin, mitomycin, methotrexate, taxanes, nilutamide, olaparib, onapristone, paclitaxel, pemetrexed, prednimustine, procarbazine, RPR109881, selumetinib, stramustine phosphate, tamoxifen, tasonermin, taxol, tretinoin, vinblastine, vincristine, vindesine sulfate, and vinflunine, and pharmaceutically acceptable salts thereof.

The additional therapeutic agent can be a vascular endothelial growth factor (VEGF) receptor inhibitors, including but not limited to, bevacizumab (sold under the trademark AVASTIN by Genentech/Roche), axitinib (described in PCT International Patent Publication No. WO01/002369), Brivanib Alaninate ((S)-((R)-1-(4-(4-Fluoro-2-methyl-1H-indol-5-yloxy)-5-methylpyrrolo[2,1-f][1,2,4]triazin-6-yloxy)propan-2-yl)2-aminopropanoate, also known as BMS-582664), motesanib (N-(2,3-dihydro-3,3-dimethyl-1H-indol-6-yl)-2-[(4-pyridinylmethyl)amino]-3-pyridinecarboxamide. and described in PCT International Patent Application Publication No. WO02/068470), pasireotide (also known as SO 230, and described in PCT International Patent Publication No. WO02/010192), and sorafenib.

The additional therapeutic agent can be a topoisomerase II inhibitor, including but not limited to, etoposide and teniposide.

The additional therapeutic agent can be an alkylating agent, including but not limited to, 5-azacytidine, decitabine, temozolomide, dactinomycin (also known as actinomycin-D, melphalan, altretamine, carmustine, bendamustine, busulfan, carboplatin, lomustine, cisplatin, chlorambucil, cyclophosphamide, dacarbazine, altretamine, ifosfamide, procarbazine, mechlorethamine, streptozocin, thiotepa, and pharmaceutically acceptable salts thereof.

The additional therapeutic agent can be an anti-tumor antibiotic, including but not limited to, doxorubicin, bleomycin, daunorubicin liposomal (daunorubicin citrate liposome), mitoxantrone, epirubicin, idarubicin, and mitomycin C.

The additional therapeutic agent can be an antimetabolite, including but not limited to, claribine, 5-fluorouracil, 6-thioguanine, cytarabine (also known as arabinosylcytosine (Ara-C)), cytarabine liposomal (also known as Liposomal Ara-C, sold under the tradename DEPOCYT™) decitabine (sold under the tradename DACOGEN®), hydroxyurea and fludarabine, floxuridine, cladribine (also known as 2-chlorodeoxyadenosine (2-CdA), methotrexate (also known as amethopterin, methotrexate sodium (MTX)), pemetrexed, and pentostatin.

The additional therapeutic agent can be a retinoid, including but not limited to, alitretinoin, tretinoin, isotretinoin, and bexarotene.

### V. Pharmaceutical Compositions

In another aspect, provided herein is a pharmaceutical composition comprising a PD-1 antagonist, an anti-ILT4 antibody or variant thereof, and a pharmaceutically acceptable excipient.

In certain embodiments, the PD-1 antagonist is an anti-PD-1 antibody. In other embodiments, the PD-1 antagonist is an anti-PD-L1 antibody.

In some embodiments, the pharmaceutical composition comprises 200 mg anti-PD-1 antibody or variant thereof, 3-1600 mg of anti-ILT4 antibody or variant thereof, and a pharmaceutically acceptable excipient, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:22, and CDR-H3 of SEQ ID NO:18, and a light chain variable region comprising light chain CDR-L1 of SEQ ID NO:11, CDR-L2 of SEQ ID NO:21, and CDR-L3 of SEQ ID NO:13.

In certain embodiments, the pharmaceutical composition comprises 200 mg anti-PD-1 antibody or variant thereof, 3-1600 mg of anti-ILT4 antibody or variant thereof, and a pharmaceutically acceptable excipient, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO: 16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In some embodiments, the pharmaceutical composition comprises 200 mg pembrolizumab or pembrolizumab variant, 3-1600 mg of anti-ILT4 antibody or variant thereof, and a pharmaceutically acceptable excipient, wherein the anti-ILT4 antibody comprises a heavy chain variable region comprising heavy chain CDR-H1 of SEQ ID NO:16, CDR-H2 of SEQ ID NO:22, and CDR-H3 of SEQ ID NO:18, and a light chain variable region comprising light chain CDR-L1 of SEQ ID NO: 11, CDR-L2 of SEQ ID NO:21, and CDR-L3 of SEQ ID NO:13.

In certain embodiments, the pharmaceutical composition comprises 200 mg pembrolizumab or pembrolizumab variant, 3-1600 mg of anti-ILT4 antibody or variant thereof, and a pharmaceutically acceptable excipient, wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO: 11, CDR-L2 of SEQ ID NO:12, and CDR-L3 of SEQ ID NO:13.

In one embodiment, the pharmaceutical composition comprises 200 mg pembrolizumab or pembrolizumab variant, 100 mg of anti-ILT4 antibody or variant thereof, and a pharmaceutically acceptable excipient, wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In another embodiment, the pharmaceutical composition comprises 200 mg pembrolizumab or pembrolizumab variant, 300 mg of anti-ILT4 antibody or variant thereof, and a pharmaceutically acceptable excipient, wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In yet another embodiment, the pharmaceutical composition comprises 200 mg pembrolizumab or pembrolizumab variant, 800 mg of anti-ILT4 antibody or variant thereof, and a pharmaceutically acceptable excipient, wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO: 11, CDR-L2 of SEQ ID NO:12, and CDR-L3 of SEQ ID NO:13.

In still another embodiment, the pharmaceutical composition comprises 200 mg pembrolizumab or pembrolizumab variant, 1600 mg of anti-ILT4 antibody or variant thereof, and a pharmaceutically acceptable excipient, wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO: 11, CDR-L2 of SEQ ID NO:12, and CDR-L3 of SEQ ID NO:13.

In some embodiments, the pharmaceutical composition comprises 200 mg pembrolizumab or pembrolizumab variant, 3-1600 mg of anti-ILT4 antibody or variant thereof, and a pharmaceutically acceptable excipient, wherein the anti-ILT4 antibody comprises a heavy chain variable region of SEQ ID NO:19 and a light chain variable region of SEQ ID NO:14.

In one embodiment, the pharmaceutical composition comprises 200 mg pembrolizumab or pembrolizumab variant, 100 mg of anti-ILT4 antibody or variant thereof, and a pharmaceutically acceptable excipient, wherein the anti-ILT4 antibody comprises a heavy chain variable region of SEQ ID NO: 19 and a light chain variable region of SEQ ID NO:14.

In another embodiment, the pharmaceutical composition comprises 200 mg pembrolizumab or pembrolizumab variant, 300 mg of anti-ILT4 antibody or variant thereof, and a pharmaceutically acceptable excipient, wherein the anti-ILT4 antibody comprises a heavy chain variable region of SEQ ID NO: 19 and a light chain variable region of SEQ ID NO:14.

In yet another embodiment, the pharmaceutical composition comprises 200 mg pembrolizumab or pembrolizumab variant, 800 mg of anti-ILT4 antibody or variant thereof, and a pharmaceutically acceptable excipient, wherein the anti-ILT4 antibody comprises a heavy chain variable region of SEQ ID NO: 19 and a light chain variable region of SEQ ID NO:14.

In still another embodiment, the pharmaceutical composition comprises 200 mg pembrolizumab or pembrolizumab variant, 1600 mg of anti-ILT4 antibody or variant thereof, and a pharmaceutically acceptable excipient, wherein the anti-ILT4 antibody comprises a heavy chain variable region of SEQ ID NO:19 and a light chain variable region of SEQ ID NO:14.

In other embodiments, the pharmaceutical composition comprises 200 mg pembrolizumab or pembrolizumab variant, 3-1600 mg of anti-ILT4 antibody or variant thereof, and a pharmaceutically acceptable excipient, wherein the anti-ILT4 antibody comprises a heavy chain of SEQ ID NO:20 and a light chain of SEQ ID NO: 15.

In one embodiment, the pharmaceutical composition comprises 200 mg pembrolizumab or pembrolizumab variant, 100 mg of anti-ILT4 antibody or variant thereof, and a pharmaceutically acceptable excipient, wherein the anti-ILT4 antibody comprises a heavy chain of SEQ ID NO:20 and a light chain of SEQ ID NO:15.

In another embodiment, the pharmaceutical composition comprises 200 mg pembrolizumab or pembrolizumab variant, 300 mg of anti-ILT4 antibody or variant thereof, and a pharmaceutically acceptable excipient, wherein the anti-ILT4 antibody comprises a heavy chain of SEQ ID NO:20 and a light chain of SEQ ID NO:15.

In yet another embodiment, the pharmaceutical composition comprises 200 mg pembrolizumab or pembrolizumab variant, 800 mg of anti-ILT4 antibody or variant thereof, and a pharmaceutically acceptable excipient, wherein the anti-ILT4 antibody comprises a heavy chain of SEQ ID NO:20 and a light chain of SEQ ID NO:15.

In still another embodiment, the pharmaceutical composition comprises 200 mg pembrolizumab or pembrolizumab variant, 1600 mg of anti-ILT4 antibody or variant thereof, and a pharmaceutically acceptable excipient, wherein the anti-ILT4 antibody comprises a heavy chain of SEQ ID NO:20 and a light chain of SEQ ID NO: 15.

### VI. Kits

In yet another aspect, provided herein is a kit for treating cancer comprising a PD-1 antagonist and an anti-ILT4 antibody or variant thereof. In one embodiment, the PD-1 antagonist is an anti-PD-1 antibody. In another embodiment, the PD-1 antagonist is an anti-PD-L1 antibody. In some embodiments, the kit further comprises instructions of use.

In certain embodiments, provided herein is a kit for treating cancer comprising 200-400 mg of anti-PD-1 antibody or variant thereof and 3-1600 mg of anti-ILT4 antibody or variant thereof, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:22, and CDR-H3 of SEQ ID NO:18, and a light chain variable region comprising light chain CDR-L1 of SEQ ID NO:11, CDR-L2 of SEQ ID NO:21, and CDR-L3 of SEQ ID NO:13.

In some embodiments, provided herein is a kit for treating cancer comprising 200-400 mg of anti-PD-1 antibody or variant thereof and 3-1600 mg of anti-ILT4 antibody or variant thereof, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In certain embodiments, provided herein is a kit for treating cancer comprising 200-400 mg pembrolizumab or pembrolizumab variant and 3-1600 mg of anti-ILT4 antibody or variant thereof, wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO: 11, CDR-L2 of SEQ ID NO:12, and CDR-L3 of SEQ ID NO:13.

In one embodiment, the kit comprises 200 mg pembrolizumab or pembrolizumab variant and 100 mg of anti-ILT4 antibody or variant thereof, wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In another embodiment, the kit comprises 200 mg pembrolizumab or pembrolizumab variant and 300 mg of anti-ILT4 antibody or variant thereof, wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In yet another embodiment, the kit comprises 200 mg pembrolizumab or pembrolizumab variant and 800 mg of anti-ILT4 antibody or variant thereof, wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In still another embodiment, the kit comprises 200 mg pembrolizumab or pembrolizumab variant and 1600 mg of anti-ILT4 antibody or variant thereof, wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In one embodiment, the kit comprises 400 mg pembrolizumab or pembrolizumab variant and 100 mg of anti-ILT4 antibody or variant thereof, wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In another embodiment, the kit comprises 400 mg pembrolizumab or pembrolizumab variant and 300 mg of anti-ILT4 antibody or variant thereof, wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In yet another embodiment, the kit comprises 400 mg pembrolizumab or pembrolizumab variant and 800 mg of anti-ILT4 antibody or variant thereof, wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In still another embodiment, the kit comprises 400 mg pembrolizumab or pembrolizumab variant and 1600 mg of anti-ILT4 antibody or variant thereof, wherein the anti-ILT4 antibody comprises a heavy chain variable region comprising heavy chain CDR-H1 of SEQ ID NO:16, CDR-H2 of SEQ ID NO: 17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

In some embodiments, the kit comprises 200-400 mg pembrolizumab or pembrolizumab variant and 3-1600 mg of anti-ILT4 antibody or variant thereof, wherein the anti-ILT4 antibody comprises a heavy chain variable region of SEQ ID NO: 19 and a light chain variable region of SEQ ID NO: 14.

In one embodiment, the kit comprises 200 mg pembrolizumab or pembrolizumab variant and 100 mg of anti-ILT4 antibody or variant thereof, wherein the anti-ILT4 antibody comprises a heavy chain variable region of SEQ ID NO: 19 and a light chain variable region of SEQ ID NO:14.

In another embodiment, the kit comprises 200 mg pembrolizumab or pembrolizumab variant and 300 mg of anti-ILT4 antibody or variant thereof, wherein the anti-ILT4 antibody comprises a heavy chain variable region of SEQ ID NO: 19 and a light chain variable region of SEQ ID NO:14.

In yet another embodiment, the kit comprises 200 mg pembrolizumab or pembrolizumab variant and 800 mg of anti-ILT4 antibody or variant thereof, wherein the anti-ILT4 antibody comprises a heavy chain variable region of SEQ ID NO: 19 and a light chain variable region of SEQ ID NO: 14.

In still another embodiment, the kit comprises 200 mg pembrolizumab or pembrolizumab variant and 1600 mg of anti-ILT4 antibody or variant thereof, wherein the anti-ILT4 antibody comprises a heavy chain variable region of SEQ ID NO: 19 and a light chain variable region of SEQ ID NO: 14.

In one embodiment, the kit comprises 400 mg pembrolizumab or pembrolizumab variant and 100 mg of anti-ILT4 antibody or variant thereof, wherein the anti-ILT4 antibody comprises a heavy chain variable region of SEQ ID NO:19 and a light chain variable region of SEQ ID NO:14.

In another embodiment, the kit comprises 400 mg pembrolizumab or pembrolizumab variant and 300 mg of anti-ILT4 antibody or variant thereof, wherein the anti-ILT4 antibody comprises a heavy chain variable region of SEQ ID NO: 19 and a light chain variable region of SEQ ID NO: 14.

In yet another embodiment, the kit comprises 400 mg pembrolizumab or pembrolizumab variant and 800 mg of anti-ILT4 antibody or variant thereof, wherein the anti-ILT4 antibody comprises a heavy chain variable region of SEQ ID NO: 19 and a light chain variable region of SEQ ID NO: 14.

In still another embodiment, the kit comprises 400 mg pembrolizumab or pembrolizumab variant and 1600 mg of anti-ILT4 antibody or variant thereof, wherein the anti-ILT4 antibody comprises a heavy chain variable region of SEQ ID NO: 19 and a light chain variable region of SEQ ID NO: 14.

In other embodiments, the kit comprises 200-400 mg pembrolizumab or pembrolizumab variant and 3-1600 mg of anti-ILT4 antibody or variant thereof, wherein the anti-ILT4 antibody comprises a heavy chain of SEQ ID NO:20 and a light chain of SEQ ID NO: 15.

In one embodiment, the kit comprises 200 mg pembrolizumab or pembrolizumab variant and 100 mg of anti-ILT4 antibody or variant thereof, wherein the anti-ILT4 antibody comprises a heavy chain of SEQ ID NO:20 and a light chain of SEQ ID NO: 15.

In another embodiment, the kit comprises 200 mg pembrolizumab or pembrolizumab variant and 300 mg of anti-ILT4 antibody or variant thereof, wherein the anti-ILT4 antibody comprises a heavy chain of SEQ ID NO:20 and a light chain of SEQ ID NO: 15.

In yet another embodiment, the kit comprises 200 mg pembrolizumab or pembrolizumab variant and 800 mg of anti-ILT4 antibody or variant thereof, wherein the anti-ILT4 antibody comprises a heavy chain of SEQ ID NO:20 and a light chain of SEQ ID NO:15.

In still another embodiment, the kit comprises 200 mg pembrolizumab or pembrolizumab variant and 1600 mg of anti-ILT4 antibody or variant thereof, wherein the anti-ILT4 antibody comprises a heavy chain of SEQ ID NO:20 and a light chain of SEQ ID NO:15.

In one embodiment, the kit comprises 400 mg pembrolizumab or pembrolizumab variant and 100 mg of anti-ILT4 antibody or variant thereof, wherein the anti-ILT4 antibody comprises a heavy chain of SEQ ID NO:20 and a light chain of SEQ ID NO: 15.

In another embodiment, the kit comprises 400 mg pembrolizumab or pembrolizumab variant and 300 mg of anti-ILT4 antibody or variant thereof, wherein the anti-ILT4 antibody comprises a heavy chain of SEQ ID NO:20 and a light chain of SEQ ID NO: 15.

In yet another embodiment, the kit comprises 400 mg pembrolizumab or pembrolizumab variant and 800 mg of anti-ILT4 antibody or variant thereof, wherein the anti-ILT4 antibody comprises a heavy chain of SEQ ID NO:20 and a light chain of SEQ ID NO:15.

In still another embodiment, the kit comprises 400 mg pembrolizumab or pembrolizumab variant and 1600 mg of anti-ILT4 antibody or variant thereof, wherein the anti-ILT4 antibody comprises a heavy chain of SEQ ID NO:20 and a light chain of SEQ ID NO:15.

### VII. General Methods

Monoclonal, polyclonal, and humanized antibodies can be prepared (see, e.g., Sheperd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fuse with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### EXAMPLES

### Example 1. Phase 1b Clinical Study of an Anti-ILT4 Antibody MAB1 as Monotherapy or in Combination with Pembrolizumab in Advanced Solid Tumors

### Study Design

MAB1 is currently being evaluated as monotherapy and in combination with pembrolizumab in an ongoing FIH, Phase 1b clinical study in participants with histologically or cytologically confirmed diagnosis of an advanced solid tumor (Table 3). Study objectives include exploring the safety, tolerability, PK, PD, and efficacy of MAB1 in combination with pembrolizumab and to establish a preliminary recommended Phase 2 dose.

An interim analysis includes safety data of all 84 participants up to the data cutoff date of 12 Mar. 2020. In the FIH Phase 1 study, Part A comprised the ATD dose escalation, Part B comprised the mTPI dose escalation, and Part C comprised the combination dose escalation. The study design mandated that each cohort in Parts A and B was opened as soon as it was confirmed that the preceding dose was safe. Part C combination was opened when 2 dose levels below the current dose of monotherapy was confirmed as safe.

TABLE 3
Summary of Study Design

| | | | Patient |
|---|---|---|---|
| | Study Part | Regimen | Population |
| Cohort 1 | A: Dose | MAB1 3 mg q3w | All solid |
| | Escalation ATD | | tumors |
| Cohort 2 | A: Dose | MAB1 10 mg q3w | All solid |
| | Escalation ATD | | tumors |
| Cohort 3 | A: Dose | MAB1 30 mg q3w | All solid |
| | Escalation ATD | | tumors |
| Cohort 1 | B: Dose | MAB1 100 mg q3w | All solid |
| | Escalation mTPI | | tumors |
| Cohort 2 | B: Dose | MAB1 300 mg q3w | All solid |
| | Escalation mTPI | | tumors |
| Cohort 3 | B: Dose | MAB1 800 mg q3w | All solid |
| | Escalation mTPI | | tumors |
| Cohort 4 | B: Dose | MAB1 1600 mg q3w | All solid |
| | Escalation mTPI | | tumors |
| Cohort 1 | C: Dose | MAB1 100 mg q3w + | All solid |
| | Escalation Combo | pembrolizumab | tumors |
| | | 200 mg q3w | |
| Cohort 2 | C: Dose | MAB1 300 mg q3w + | All solid |
| | Escalation Combo | pembrolizumab | tumors |
| | | 200 mg q3w | |
| Cohort 3 | C: Dose | MAB1 800 mg q3w + | All solid |
| | Escalation Combo | pembrolizumab | tumors |
| | | 200 mg q3w | |
| Cohort 4 | C: Dose | MAB1 1600 mg q3w + | All solid |
| | Escalation Combo | pembrolizumab | tumors |
| | | 200 mg q3w | |

Abbreviations: ATD = accelerated titration dose; q3w = every 3 weeks; mTPI = modified toxicity probability interval.

### Pharmacokinetics and Product Metabolism in Humans

A validated electrochemiluminescence immunoassay was used to assess MAB1 concentrations in human serum. The method utilizes a pair of anti-MAB1 antibodies (generated in house) as capture and detection reagents. MAB1 present in human serum samples is captured by the formation of a complex between biotinylated anti-MAB1 and ruthenylated anti-MAB1 antibody. Unbound material is removed by plate washing, MSD read buffer (Meso Scale Discovery, Rockville, MD) is added, and the bound complexes are detected by electrochemiluminescence on MESO SECTOR S 600 (Meso Scale Discovery). The lower limit of quantitation of the assay is 81.2 ng/mL and the minimum required dilution (MRD) is 50. This PK assay was validated in accordance with current regulatory guidance.

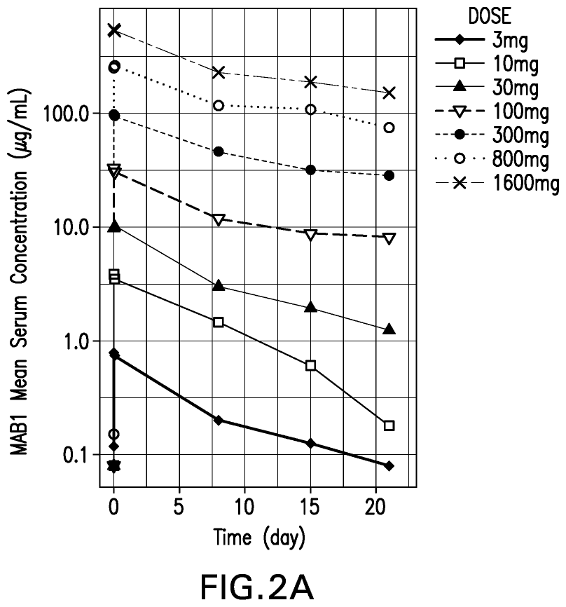

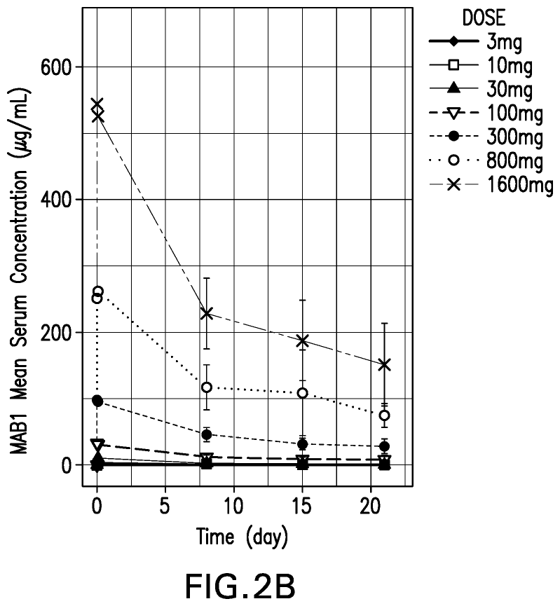

Preliminary PK data from patients treated during dose escalation (MAB1 alone or in combination with pembrolizumab) at doses from 3 mg to 1600 mg showed that serum MAB1 exposures increased in a dose-dependent manner (FIGS. 2A and 2B).

### ILT4 Receptor Occupancy in Whole Blood

A flow cytometric ILT4 membrane receptor occupancy (RO) assay was developed and fit-for-purpose validated in Streck Cytochex® BCT collected whole blood to support exploratory pharmacodynamic (target engagement) endpoints. A dual detection assay method is employed to measure total and unoccupied (free) ILT4 membrane receptor expression on total monocytes in whole blood collected from patients at pre- and post-MAB treatment timepoints. Using this RO assay format, one detection antibody will compete with the epitope targeted by MAB1 (to detect unoccupied/free receptor) while the other detection antibody will recognize another epitope that does not compete with MAB1 (to detect total receptor expression).

Key reagents for the whole blood RO assay are shown in Table 4. The assay is performed in 100 μL of whole blood taken from a 1×5 mL Streck CytoChex® BCT. Whole blood is treated with an Fc blocking agent for 10 minutes and stained with the FULL panel (Table 5) or FMX control panel (Table 6) at 2-8BC for 30 minutes. Red blood cells are lysed using 2×BD FACS lysing solution for 15 minutes. Cells are then washed before a final resuspension in 2 fetal bovine serum. Labeled cells are acquired on a BD LSR flow cytometer equipped with 5 lasers using BD FACSDiva software v.8.0.1. The BD LSR instrument configuration is shown in Table 7. Data analysis is performed offline with FlowJo software v.9.9. A hierarchal gating strategy is applied to identify total monocytes encompassing classical, intermediate, and non-classical subsets. The phenotypic definition of total monocytes is shown in Table 8. The percent ILT4+APC (unoccupied) and the percent ILT4+PE (total) gate is set to ≤0.5 in the FMX stained sample to identify HLT4+ monocytes in the FULL stained sample. Percent ILT4 membrane receptor occupancy is calculated as follows:

Percent ILT4Membrane Receptor Occupancy=[1−(% ILT4+_{unoccupied}÷% ILT4+ₜₒₜₐₗ)]×100%.

TABLE 4
Key reagents

| Product Name | Company | Catalogue |
|---|---|---|
| Human BD FC Block | BD Biosciences | 564220 |
| BD FACS Lysing Solution | BD Biosciences | 349202 |
| BUV737 Mouse Anti-Human CD19 | BD Biosciences | 564303 |
| BUV737 Mouse Anti-Human CD56 | BD Biosciences | 564447 |
| BUV737 Mouse Anti-Human CD3 | BD Biosciences | 564307 |
| Brilliant Violet 650 Anti-Human | Biolegend | 301336 |
| CD11b | | |
| PE/Cy7 Anti-Human CD14 | Biolegend | 301814 |
| APC-eFlour 780 CD16 | Thermo Fisher | 47-0168-42 |
| Monoclonal Antibody | Scientific | |
| BB615 Mouse Anti-Human CD33 | BD Biosciences | 564588 |
| BV786 Mouse Anti-Human CD45 | BD Biosciences | 563716 |
| BV421 Mouse Anti-Human CD66b | BD Biosciences | 562940 |
| BUV395 Mouse Anti-Human | BD Biosciences | 564040 |
| HLA-DR | | |
| Human LILRB2/CD85d/ILT4 PE | R&D Systems | FAB2078P-100 |

TABLE 5
Whole Blood ILT4 Receptor Occupancy Full Panel Composition
Whole Blood FULL Panel

| Target | Fluorochrome | Clone | Titer |
|---|---|---|---|
| CD3 | BUV737 | UCHT1 | 1:200 |
| CD19 | BUV737 | SJC25C1 | 1:100 |
| CD56 | BUV737 | NCAM16.2 | 1:25 |
| CD11b | Brilliant Violet | ICRF44 | 1:50 |
| | 650 | | |
| CD14 | PE/Cy7 | M5E2 | 1:100 |
| CD16 | APC-eFluor 780 | eBioCB16 | 1:200 |
| | | (CB16) | |
| CD33 | BB515 | WM53 | 1:25 |
| CD45 | BV786 | HI30 | 1:50 |
| CD66b | BV421 | G10F5 | 1:100 |
| HLA-DR | BUV395 | G46-6 | 1:25 |
| ILT4 (unoccupied) | APC | 14ASS | 1:200 |
| ILT4 (total) | PE | 287219 | 1:25 |

TABLE 6
Whole Blood ILT4 Receptor Occupancy FMX Control Panel Composition
Whole Blood FMX Control Panel

| Target | Fluorochrome | Clone | Titer |
|---|---|---|---|
| CD3 | BUV737 | UCHT1 | 1:200 |
| CD19 | BUV737 | SJC25C1 | 1:100 |
| CD56 | BUV737 | NCAM16.2 | 1:25 |
| CD11b | Brilliant Violet | ICRF44 | 1:50 |
| | 650 | | |
| CD14 | PE/Cy7 | M5E2 | 1:100 |
| CD16 | APC-eFluor 780 | eBioCB16 | 1:200 |
| | | (CB16) | |
| CD33 | BB515 | WM53 | 1:25 |
| CD45 | BV786 | HI30 | 1:50 |
| CD66b | BV421 | G10F5 | 1:100 |
| HLA-DR | BUV395 | G46-6 | 1:25 |
| ILT4 (unoccupied) | N/Ap | N/Ap | — |
| ILT4 (total) | N/Ap | N/Ap | — |

TABLE 7
Summary of BD LSR Flow Cytometer Configuration

| | | | Detector | | |
|---|---|---|---|---|---|
| Laser | Wavelength | Power | Array | Mirror | Filter |
| Blue | 488 nm | 50 mW | Octagon | 630 LP | 695/40 BP |
| | | | | 505 LP | 530/30 BP |
| | | | | | 488/10 BP |
| Violet | 405 nm | 50 mW | Octagon | 750 LP | 780/60 BP |
| | | | | 690 LP | 710/50 BP |
| | | | | 630 LP | 660/20 BP |
| | | | | 595 LP | 610/20 BP |
| | | | | 505 LP | 525/50 BP |
| | | | | | 450/50 BP |
| Yellow | 561 nm | 50 mW | Octagon | 735 LP | 780/60 BP |
| Green | | | | 635 LP | 670/30 BP |
| | | | | 600 LP | 610/20 BP |
| | | | | | 585/15 BP |
| Red | 640 nm | 450 mW | Trigon | 735 LP | 780/60 BP |
| | | | | 710 LP | 730/45 BP |
| | | | | | 670/45 BP |
| UV | 355 nm | 20 mW | Trigon | 690 LP | 740/35 BP |
| | | | | 450 LP | 515/30 BP |
| | | | | | 379/25 BP |

TABLE 8
Phenotypic Description of Whole Blood Total Monocytes

| | | CD3/ | | | | | | |
|---|---|---|---|---|---|---|---|---|
| Population | Subset | CD19/CD56 | CD11b | CD14 | CD16 | CD66b | HLA-DR | CD33 |
| Monocytes | Total | − | + | +/− | +/− | − | + | + |

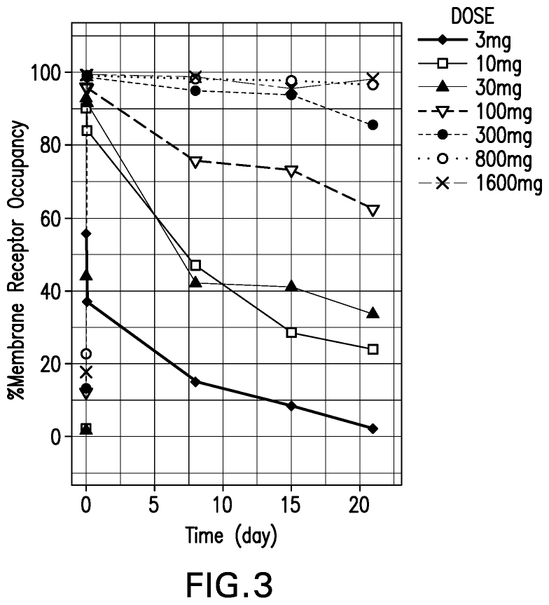

Preliminary data for the serum target engagement PD marker from patients treated during dose escalation (MAB1 alone and in combination with pembrolizumab) at doses from 3 mg to 1600 mg also showed a dose-dependent increase in membrane receptor occupancy in serum (FIG. 3). The average membrane target occupancy was >90% at 800 mg or higher doses.

### Anti-Drug Antibody (ADA) Summary

A validated electrochemiluminescence immunoassay was used to assess ADA responses to MAB1 in human serum. Human serum samples and controls were first incubated with 300 mM acetic acid at room temperature to free anti-MAB1 (ADA) from all non-specific or specific binding partners. Acid-treated samples were then neutralized with 300 mM Tris buffer (pH 9.5) containing biotinylated MAB1. After incubation with the biotinylated MAB1 at room temperature for 1 hour, the sample mixture was transferred to MSD Streptavidin Gold Plate (Meso Scale Discovery) pre-blocked with 1.0% BSA/0.05% PBST, where the biotinylated MAB1 in the complex bound to the streptavidin in the wells. Then, after incubation at room temperature for 1 hour, the unbound materials were removed by washing the plate and a ruthenylated MAB1 (SULFO-TAG™ MAB1) was used as detection. After incubation and washing, MSD read buffer (Meso Scale Discovery) was added and the bound ADA complexes were detected by reading electrochemiluminescence signals using MESO SECTOR S 600 (Meso Scale Discovery). The ADA assay was validated in accordance with current regulatory guidance.

The immunogenicity assessment for MAB1 included all available ADA sample results from participants who had at least one ADA sample available after dosing with MAB1. A total of 80 participants were evaluable for MAB1 immunogenicity assessment. No serum ADA samples have been confirmed positive for anti-MAB1 antibodies.

### Efficacy

As of 12 Mar. 2020, preliminary efficacy data from participants dosed with MAB1 were available from 50 participants dosed in monotherapy and 34 participants dosed in MAB1 in combination therapy with pembrolizumab.

Preliminary data show that, of the 50 participants treated with MAB1 monotherapy in the initial treatment phase (FAS population), the best overall response (with response confirmation) was a PR for 1 participant (2%). The time to response for this participant was 17.6 weeks and the duration of response was greater than 9 weeks. In addition, 11 participants (22%) treated with MAB1 monotherapy experienced SD.

Preliminary data also show that, of the 34 participants treated with MAB1 and pembrolizumab combination therapy in the initial treatment phase (FAS population), the BOR (with response confirmation) was PR for 7 participants (20.6%). The median time to response was 17.1 weeks (range: 8.1-21.7 weeks) for these participants. The duration of response ranged from greater than 9.1 weeks to greater than 27.3 weeks; 6 participants had an extended response duration of >12 weeks, of whom 2 had a duration of response greater than >24 weeks. In addition, 10 participants (29.4%) treated with MAB1 and pembrolizumab combination therapy experienced SD.

Additionally, 17 participants initially treated with MAB1 monotherapy (FAS population) crossed over to receive either pembrolizumab alone (1 participant) or to receive MAB1 and pembrolizumab combination therapy (16 participants). Of these 17 patients, 12 participants did not yet have an efficacy assessment at the time of the data cutoff. The best overall response for the rest 5 participants included 3 participants with PD, 1 participant with SD (MAB1 1600 mg+ pembrolizumab), and 1 participant who was nonevaluable.

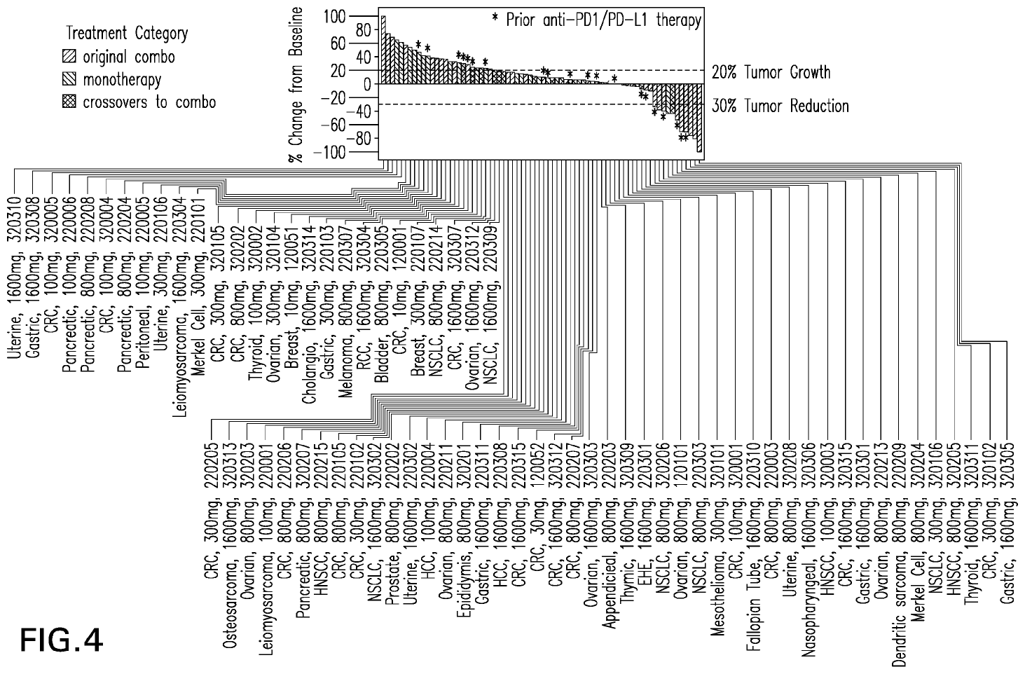

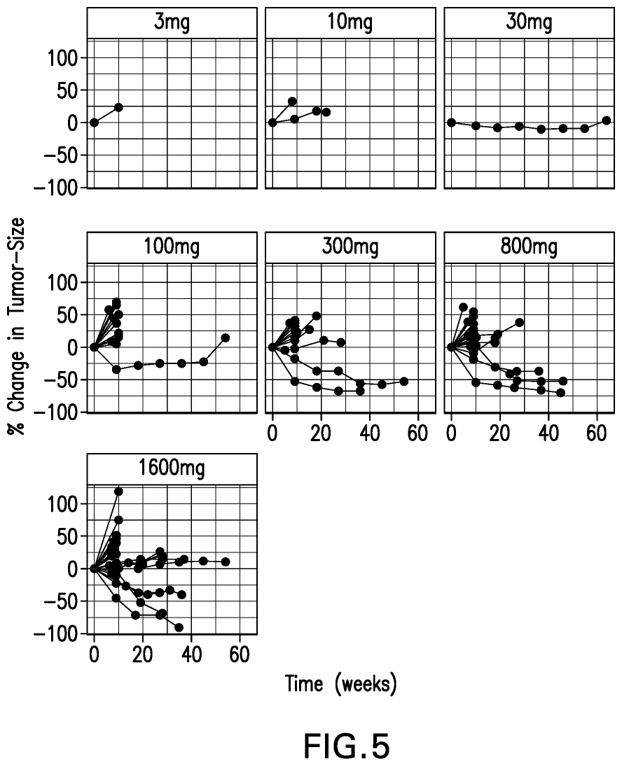

The efficacy of treatment with MAB1 alone or in combination with pembrolizumab in various solid tumors is shown in a waterfall plot (FIG. 4). Percentage of tumor size change in various solid tumors is sorted by dosage (FIG. 5).

### Safety

At the time of the 12 Mar. 2020 data cutoff, a total of 84 participants had received MAB1; 50 participants received MAB1 monotherapy up to the maximum planned dose level of 1600 mg and 34 participants received MAB1 (up to 1600 mg) and pembrolizumab combination therapy. An additional 19 patients (ASaT population) who had received monotherapy crossed over to receive either pembrolizumab (1) or combination therapy (18).

Of the 84 participants administered MAB1 as of 12 Mar. 2020, approximately half were female (51.2%), the majority were white (86.9%), and the population had a median age of 62 years (range 31 to 83). All participants had received prior lines of therapy (one line: 16.7%; two lines: 28.6%; three lines: 16.7%; four lines: 13.1%; five lines or greater: 2.2%; missing: 2.4%). The baseline characteristics for participants who received monotherapy and participants who initially received combination therapy were similar (although slightly fewer participants in the monotherapy group had an ECOG-PS of 1 [monotherapy: 53.3%; combination therapy: 61.8%] and slightly fewer were Hispanic [monotherapy: 4.0%; combination therapy: 14.7%]).

Of the 84 participants who started study intervention, 66 had discontinued study intervention:
52 (61.9%) due to progressive disease (monotherapy: 36/50, 72.0%; combination therapy: 16/34, 47.1%),
11 (13.1%) due to clinical progression (monotherapy: 6/50, 12.0%; combination therapy: 5/34, 14.7%),
2 (2.4%) due to AEs of aspartate aminotransferase increased and blood bilirubin increased (Sjorgen's Syndrome) (monotherapy: 2/50, 2.4%; combination therapy: 0),
1 (1.2%) due to withdrawal by participant (monotherapy: 1/50, 2.0%; combination therapy: 0).

As of the data cutoff (12 Mar. 2020), 83 of 84 participants (98.8%) had experienced 1 or more AEs, of whom 44 participants (52.4%) experienced AE(s) that were considered by the investigator to be related to study intervention (Table 9). Thirty participants (35.7%) experienced SAEs as of the data cutoff, of these, 4 participants (4.8%), all in the combination therapy group, experienced 5 SAEs (Grade 3 fatigue, Grade 2 colitis, and Grade 3 diarrhea in 1 participant each, and Grade 3 pneumonia and Grade 3 pneumonitis in 1 participant) that were considered by the investigator to be related to study intervention. Grade 3-5 AEs were reported for 44 participants (52.4%); of these, 7 participants (8.3%) experienced Grade 3-5 AEs that were considered by the investigator to be related to study intervention. One participant (1.2%) in the MAB1 monotherapy group discontinued treatment due to a drug-related AE (aspartate aminotransferase increased). Two deaths (due to mouth hemorrhage and “death”) were reported, both in the MAB1 monotherapy group and both considered by investigator as not related to study intervention (Table 9). There were no DLTs reported by the investigator as of the data cutoff.

A summary of drug-related AEs occurring in more than 1 participant by descending incidence (Table 10) shows that the most commonly occurring AEs overall (occurring in >5% of participants) included fatigue (15.5%), arthralgia (8.3%), diarrhea, hypothyroidism, nausea, and rash maculo-papular (7.1% each), and pruritis (6.0%). For the MAB1 monotherapy group, the most commonly occurring AEs (occurring in >5% of participants) included fatigue (12.0%), diarrhea (10.0%), arthralgia (8.0%), nausea, decreased appetite, aspartate aminotransferase increased, and pruritis (6.0% each). For the combination therapy group, the most commonly occurring AEs (occurring in >5% of participants) included fatigue (13.5%), hypothyroidism and rash maculo-papular (7.7% each), as well as arthralgia and nausea (5.8% each). Grade 3 drug-related AEs that occurred in more than 1 participant included fatigue and aspartate aminotransferase increased, which occurred in 2 participants each. No drug-related AEs >Grade 3 were reported.

There were 16 participants with AEs of special interest during this reporting period, including 4 in the MAB1 monotherapy group (hypothyroidism [2], infusion-related reaction [1], and rash maculo-papular [1]), and 12 in the MAB1 and pembrolizumab combination therapy group (hypothyroidism [6], pneumonitis [2], infusion-related reaction [1], hypersensitivity [1], hyperthyroidism [1], and colitis [1]).

TABLE 9
Adverse Event Summary - ASaT Population

| | MAB1 | | MAB1 + | | | |
|---|---|---|---|---|---|---|
| | Monotherapy | | Pembrolizumab | | Total | |
| | N = 50 | | N = 52 ^{a} | | N = 84 | |
| Subjects in population: | n (%) | | n (%) | | n (%) | |
| with 1 or more AEs | 50 | (100.0) | 47 | (90.4) | 83 | (98.8) |
| with drug-related AEs ^{b} | 23 | (46.0) | 27 | (51.9) | 44 | (52.4) |
| with toxicity Grade 3-5 | 23 | (46.0) | 24 | (46.2) | 44 | (52.4) |
| AEs | | | | | | |
| with toxicity Grade 3-5 | 3 | (6.0) | 4− | (7.7) | 7 | (8.3) |
| drug-related AEs ^{b} | | | | | | |
| with SAEs | 14 | (28.0) | 18 | (34.6) | 30 | (35.7) |

| | | | | | |
|---|---|---|---|---|---|
| with drug-related SAEs ^{b} | 0 | 4 | (7.7) | 4 | (4.8) |

| who died | 2 | (4.0) | 0 | 2 | (2.4) |
|---|---|---|---|---|---|
| who died due to a | 0 | | 0 | 0 | |
| drug-related AE ^{a} | | | | | |
| discontinued drug due to | 2 | (4.0) | 0 | 2 | (2.4) |
| an AE | | | | | |
| discontinued drug due to a | 1 | (2.0) | 0 | 1 | (1.2) |
| drug-related AE ^{a} | | | | | |

| | | | |
|---|---|---|---|
| discontinued drug due to | 0 | 0 | 0 |
| an SAE | | | |
| discontinued drug due to a | 0 | 0 | 0 |
| drug-related SAE ^{a} | | | |

^{a} Includes participants who crossed over from monotherapy to combination therapy.
^{b} Determined by the investigator to be related to the drug.
Non-serious AEs up to 30 days of last dose and SAEs up to 90 days of last dose are included.
MedDRA preferred terms “Neoplasm progression”, “Malignant neoplasm progression” and “Disease progression” not related to the drug are excluded.
Grades are based on NCI CTCAE version 4.0.
For participants who crossed over from MAB1 monotherapy to combination therapy, AEs reported above are as follows: For AEs that occurred prior to crossover, the information is integrated into the original MAB1 monotherapy column. For AEs that occurred subsequent to crossover, the information is integrated into the combination therapy (MAB1 + Pembrolizumab). AEs that occurred while subjects were on pembrolizumab alone are not reported. For back-fill subjects, the AEs are reported under the assigned dose cohort.
AE = adverse event; CTCAE = Common Terminology Criteria for AEs; MedDRA = Medical Dictionary for Regulatory Activities; NCI = National Cancer Institute; SAE = serious AE.

TABLE 10
Participants With Drug-related Adverse Events By Decreasing
Incidence (Occurring in >1 Participant) - ASaT Population

| | MAB1 | | MAB1 + | | | |
|---|---|---|---|---|---|---|
| | Monotherapy | | Pembrolizumab | | Total | |
| | N = 50 | | N = 52 ^{a} | | N = 84 | |
| | n (%) | | n (%) | | n (%) | |
| Fatigue | 6 | (12.0) | 7 | (13.5) | 13 | (15.5) |
| Arthralgia | 4 | (8.0) | 3 | (5.8) | 7 | (8.3) |
| Diarrhea | 5 | (10.0) | 2 | (3.8) | 6 | (7.1) |
| Hypothyroidism | 2 | (4.0) | 4 | (7.7) | 6 | (7.1) |
| Nausea | 3 | (6.0) | 3 | (5.8) | 6 | (7.1) |
| Rash maculo-papular | 2 | (4.0) | 4 | (7.7) | 6 | (7.1) |
| Pruritus | 3 | (6.0) | 2 | (3.8) | 5 | (6.0) |
| Aspartate | 3 | (6.0) | 1 | (1.9) | 4 | (4.8) |
| aminotransferase | | | | | | |
| increased | | | | | | |
| Decreased appetite | 3 | (6.0) | 1 | (1.9) | 4 | (4.8) |
| Influenza like illness | 2 | (4.0) | 1 | (1.9) | 3 | (3.6) |
| Vomiting | 2 | (4.0) | 1 | (1.9) | 3 | (3.6) |
| Anemia | 1 | (2.0) | 1 | (1.9) | 2 | (2.4) |
| Asthenia | 1 | (2.0) | 1 | (1.9) | 2 | (2.4) |
| Pneumonitis | 0 | | 2 | (3.8) | 2 | (2.4) |
| Rash | 1 | (2.0) | 1 | (1.9) | 2 | (2.4) |
| Weight decreased | 1 | (2.0) | 1 | (1.9) | 2 | (2.4) |

^{a} Includes participants who crossed over from monotherapy to combination therapy.
NOTE:
Every subject is counted a single time for each applicable specific adverse event.
A specific adverse event appears on this report only if its incidence in one or more of the columns meets the incidence criterion in the report title, after rounding.
Two AEs for 2 participants (1 per participant) was not encoded at the time of the data extraction and therefore are not reported in the table above.
Non-serious adverse events up to 30 days of last dose and serious adverse events up to 90 days of last dose are included.
MedDRA preferred terms “Neoplasm Progression” and “Malignant Neoplasm Progression” not related to the drug are excluded.
For participants who crossed over from MAB1 monotherapy to combination therapy, AEs reported above are as follows: For AEs that occurred prior to crossover, the information is integrated into the original MAB1 monotherapy column. For AEs that occurred subsequent to crossover, the information is integrated into the combination therapy (MAB1 + Pembrolizumab). AEs that occurred while participants were on pembrolizumab alone are not reported.
For back-fill subjects, the AEs are reported under the assigned dose cohort.
AE = adverse event; MedDRA = Medical Dictionary for Regulatory Activities.

All references cited herein are incorporated by reference to the same extent as if each individual publication, database entry (e.g. Genbank sequences or GeneTD entries), patent application, or patent, was specifically and individually indicated to be incorporated by reference. This statement of incorporation by reference is intended by Applicant, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

### SEQUENCE LISTING

The present specification is being filed with a computer readable form (CRF) copy of the Sequence Listing. The CRY entitled 25104_US_PSP_SEQLIST_ST25.txt, which was created on Sep. 17, 2020 and is 20 KB in size, is incorporated herein by reference in its entirety.

Table 11 below summarizes all sequences disclosed in the present specification.

TABLE 11
SEQ ID NOS and Corresponding Sequences

| SEQ | Artificial | | |
|---|---|---|---|
| ID | Sequence | Feature | |
| NO | Description | (residue #-feature info) | Amino Acid Sequence |
| 1 | Pembrolizumab, | | RASKGVSTSGYSYLH |
| | VL-CDR1 | | |
| 2 | Pembrolizumab, | | LASYLES |
| | VL-CDR2 | | |
| 3 | Pembrolizumab, | | QHSRDLPLT |
| | VL-CDR3 | | |
| 4 | Pembrolizumab, | | EIVLTQSPATLSLSPGERATLSCRASKGVST |
| | VL | | SGYSYLHWYQQKPGQAPRLLIYLASYLESGV |
| | | | PARFSGSGSGTDFTLTISSLEPEDFAVYYCQ |
| | | | HSRDLPLTFGGGTKVEIK |
| 5 | Pembrolizumab, | | EIVLTQSPATLSLSPGERATLSCRASKGVST |
| | light chain | | SGYSYLHWYQQKPGQAPRLLIYLASYLESGV |
| | | | PARFSGSGSGTDFTLTISSLEPEDFAVYYCQ |
| | | | HSRDLPLTFGGGTKVEIKRTVAAPSVFIFPP |
| | | | SDEQLKSGTASVVCLLNNFYPREAKVQWKVD |
| | | | NALQSGNSQESVTEQDSKDSTYSLSSTLTLS |
| | | | KADYEKHKVYACEVTHQGLSSPVTKSFNRGE |
| | | | C |
| 6 | Pembrolizumab, | | NYYMY |
| | VH-CDR1 | | |
| 7 | Pembrolizumab, | | GINPSNGGTNFNEKFKN |
| | VH-CDR2 | | |
| 8 | Pembrolizumab, | | RDYRFDMGFDY |
| | VH-CDR3 | | |
| 9 | Pembrolizumab, | | QVQLVQSGVEVKKPGASVKVSCKASGYTFTN |
| | VH | | YYMYWVRQAPGQGLEWMGGINPSNGGTNFNE |
| | | | KFKNRVTLTTDSSTTTAYMELKSLQFDDTAV |
| | | | YYCARRDYRFDMGFDYWGQGTTVTVSS |
| 10 | Pembrolizumab, | | QVQLVQSGVEVKKPGASVKVSCKASGYTFTN |
| | heavy chain | | YYMYWVRQAPGQGLEWMGGINPSNGGTNFNE |
| | | | KFKNRVTLTTDSSTTTAYMELKSLQFDDTAV |
| | | | YYCARRDYRFDMGFDYWGQGTTVTVSSASTK |
| | | | GPSVFPLAPCSRSTSESTAALGCLVKDYFPE |
| | | | PVTVSWNSGALTSGVHTFPAVLQSSGLYSLS |
| | | | SVVTVPSSSLGTKTYTCNVDHKPSNTKVDKR |
| | | | VESKYGPPCPPCPAPEFLGGPSVFLFPPKPK |
| | | | DTLMISRTPEVTCVVVDVSQEDPEVQFNWYV |
| | | | DGVEVHNAKTKPREEQFNSTYRVVSVLTVLH |
| | | | QDWLNGKEYKCKVSNKGLPSSIEKTISKAKG |
| | | | QPREPQVYTLPPSQEEMTKNQVSLTCLVKGF |
| | | | YPSDIAVEWESNGQPENNYKTTPPVLDSDGS |
| | | | FFLYSRLTVDKSRWQEGNVFSCSVMHEALHN |
| | | | HYTQKSLSLSLGK |
| 11 | MAB1, VL- | | TGSSSNIGAGYDVH |
| | CDR1 | | |
| 12 | MAB1, VL- | | GDSNRPS |
| | CDR2 | | |
| 13 | MAB1, VL- | | QSFDNSLSAYV |
| | CDR3 | | |
| 14 | MAB1, VL | | ESVLTQPPSVSGAPGQRVTISCTGSSSNIGA |
| | | | GYDVHWYQQLPGTAPKLLIYGDSNRPSGVPD |
| | | | RFSVSKSGASASLAITGLQAEDEADYYCQSF |
| | | | DNSLSAYVFGGGTQLTVL |
| 15 | MAB1, light | | ESVLTQPPSVSGAPGQRVTISCTGSSSNIGA |
| | chain | | GYDVHWYQQLPGTAPKLLIYGDSNRPSGVPD |
| | | | RFSVSKSGASASLAITGLQAEDEADYYCQSF |
| | | | DNSLSAYVFGGGTQLTVLGQPKAAPSVTLFP |
| | | | PSSEELQANKATLVCLISDFYPGAVTVAWKA |
| | | | DSSPVKAGVETTTPSKQSNNKYAASSYLSLT |
| | | | PEQWKSHRSYSCQVTHEGSTVEKTVAPTECS |
| 16 | MAB1, VH- | | GYYWS |
| | CDR1 | | |
| 17 | MAB1, VH- | | EINHAGSTNYNPSLKS |
| | CDR2 | | |
| 18 | MAB1, VH- | | LPTRWVTTRYFDL |
| | CDR3 | | |
| 19 | MAB1, VH | | EVQLQQWGAGLLKPSETLSLTCAVYGGSFSG |
| | | | YYWSWIRQPPGKGLEWIGEINHAGSTNYNPS |
| | | | LKSRVTISVDTSKNQFSLKLSSVTAADTAVY |
| | | | YCARLPTRWVTTRYFDLWGRGTLVTVSS |
| 20 | MAB1, heavy | | EVQLQQWGAGLLKPSETLSLTCAVYGGSFSG |
| | chain | | YYWSWIRQPPGKGLEWIGEINHAGSTNYNPS |
| | | | LKSRVTISVDTSKNQFSLKLSSVTAADTAVY |
| | | | YCARLPTRWVTTRYFDLWGRGTLVTVSSAST |
| | | | KGPSVFPLAPCSRSTSESTAALGCLVKDYFP |
| | | | EPVTVSWNSGALTSGVHTFPAVLQSSGLYSL |
| | | | SSVVTVPSSSLGTKTYTCNVDHKPSNTKVDK |
| | | | RVESKYGPPCPPCPAPEFLGGPSVFLFPPKP |
| | | | KDTLMISRTPEVTCVVVDVSQEDPEVQFNWY |
| | | | VDGVEVHNAKTKPREEQFNSTYRVVSVLTVL |
| | | | HQDWLNGKEYKCKVSNKGLPSSIEKTISKAK |
| | | | GQPREPQVYTLPPSQEEMTKNQVSLTCLVKG |
| | | | FYPSDIAVEWESNGQPENNYKTTPPVLDSDG |
| | | | SFFLYSRLTVDKSRWQEGNVFSCSVMHEALH |
| | | | NHYTQKSLSLSLGK |
| 21 | Anti-ILT4 | X₁ is N, Q, E or D | GX1X2NRPS |
| | antibody, VL- | X₂ is S or A | |
| | CDR2 consensus | | |
| | sequence | | |
| 22 | Anti-ILT4 | X is S or A | EINHXGSTNYNPSLKS |
| | antibody, VH- | | |
| | CDR2 consensus | | |
| | sequence | | |

1. A method for treating cancer in a patient comprising administering to the patient 3-1600 mg of an anti-ILT4 antibody, wherein the anti-ILT4 antibody comprises: (a) a light chain variable domain comprising CDR-L1 of SEQ ID NO:11, CDR-L2 of SEQ ID NO:21, and CDR-L3 of SEQ ID NO:13; and (b) a heavy chain variable domain comprising CDR-H1 of SEQ ID NO:16, CDR-H2 of SEQ ID NO:22, and CDR-H3 of SEQ ID NO:18.

2-12. (canceled)

13. The method of claim 1, wherein the anti-ILT4 antibody is co-administered with a PD-1 antagonist.

14. The method of claim 1, wherein the anti-ILT4 antibody is co-formulated with a PD-1 antagonist.

15. The method of claim 13, wherein the PD-1 antagonist is an anti-PD-1 antibody or antigen binding fragment thereof.

16. The method of claim 13, wherein the PD-1 antagonist is an anti-PD-L1 antibody or antigen binding fragment thereof.

17-19. (canceled)

20. The method of claim 15, wherein the anti-PD-1 antibody is pembrolizumab or a pembrolizumab variant.

21. The method of claim 15, wherein the anti-PD-1 antibody is nivolumab or cemiplimab.

22-23. (canceled)

24. The method of claim 16, wherein the anti-PD-L1 antibody is atezolizumab, durvalumab, or avelumab.

25. The method of claim 15, wherein 200 mg of the anti-PD-1 antibody is administered via intravenous infusion on Day 1 and then once every about three weeks thereafter or 400 mg of the anti-PD-1 antibody is administered via intravenous infusion on Day 1 and then once every about six weeks thereafter.

26. (canceled)

27. A method for treating cancer in a patient comprising administering to the patient 3-1600 mg of an anti-ILT4 antibody and 200-400 mg of an anti-PD-1 antibody, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO: 11, CDR-L2 of SEQ ID NO:12, and CDR-L3 of SEQ ID NO:13.

28-29. (canceled)

30. The method of claim 27, wherein 200 mg of the anti-PD-1 antibody is administered via intravenous infusion on Day 1 and then once every about three weeks thereafter, and 300 mg of the anti-ILT4 antibody is administered via intravenous infusion on Day 1 and then once every about three weeks thereafter.

31. The method of claim 27, wherein 400 mg of the anti-PD-1 antibody is administered via intravenous infusion on Day 1 and then once every about six weeks thereafter, and 300 mg of the anti-ILT4 antibody is administered via intravenous infusion on Day 1 and then once every about three weeks thereafter.

32. The method of claim 27, wherein 200 mg of the anti-PD-1 antibody is administered via intravenous infusion on Day 1 and then once every about three weeks thereafter, and 800 mg of the anti-ILT4 antibody is administered via intravenous infusion on Day 1 and then once every about three weeks thereafter.

33. The method of claim 27, wherein 400 mg of the anti-PD-1 antibody is administered via intravenous infusion on Day 1 and then once every about six weeks thereafter, and 800 mg of the anti-ILT4 antibody is administered via intravenous infusion on Day 1 and then once every about three weeks thereafter.

34. The method of claim 27, wherein 200 mg of the anti-PD-1 antibody is administered via intravenous infusion on Day 1 and then once every about three weeks thereafter, and 1600 mg of the anti-ILT4 antibody is administered via intravenous infusion on Day 1 and then once every about three weeks thereafter.

35. The method of claim 27, wherein 400 mg of the anti-PD-1 antibody is administered via intravenous infusion on Day 1 and then once every about six weeks thereafter, and 1600 mg of the anti-ILT4 antibody is administered via intravenous infusion on Day 1 and then once every about three weeks thereafter.

36-37. (canceled)

38. The method of claim 1, wherein the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer, non-small cell lung cancer, pleural mesothelioma, gastric cancer, colorectal cancer, cervical cancer, synovial sarcoma, head and neck cancer, squamous cell carcinoma, lymphoma, diffuse large B-cell lymphoma, non-Hodgkin lymphoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelofibrosis, soft tissue sarcoma, thyroid cancer, endometrial cancer, and carcinoid cancer.

39. The method of claim 1, wherein the cancer is head and neck squamous cell cancer, gastric cancer, pancreatic cancer, glioblastoma, renal cell carcinoma, or non-small cell lung cancer.

40. A pharmaceutical composition comprising 200-400 mg of an anti-PD-1 antibody or an anti-PD-1 antibody variant, 3-1600 mg of an anti-ILT4 antibody or an anti-ILT4 antibody variant, and a pharmaceutically acceptable excipient, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

41-47. (canceled)

48. A kit for treating cancer comprising 200-400 mg anti-PD-1 antibody or anti-PD-1 antibody variant and 3-1600 mg of anti-ILT4 antibody or an anti-ILT4 antibody variant, wherein the anti-PD-1 antibody comprises a heavy chain variable region comprising heavy chain CDR-H1 of SEQ ID NO:6, CDR-H2 of SEQ ID NO:7, and CDR-H3 of SEQ ID NO:8, and a light chain variable region comprising light chain CDR-L1 of SEQ ID NO:1, CDR-L2 of SEQ ID NO:2, and CDR-L3 of SEQ ID NO:3, and wherein the anti-ILT4 antibody comprises a heavy chain variable region comprising heavy chain CDR-H1 of SEQ ID NO:16, CDR-H2 of SEQ ID NO:17, and CDR-H3 of SEQ ID NO:18, and a light chain variable region comprising light chain CDR-L1 of SEQ ID NO:11, CDR-L2 of SEQ ID NO:12, and CDR-L3 of SEQ ID NO:13.

49-55. (canceled)


SEQUENCE LISTING (22 sequences)

SEQ ID NO: 1 - protein, 15 aa, Artificial Sequence
  Pembrolizumab, VL-CDR1
RASKGVSTSGYSYLH

SEQ ID NO: 2 - protein, 7 aa, Artificial Sequence
  Pembrolizumab, VL-CDR2
LASYLES

SEQ ID NO: 3 - protein, 9 aa, Artificial Sequence
  Pembrolizumab, VL-CDR3
QHSRDLPLT

SEQ ID NO: 4 - protein, 111 aa, Artificial Sequence [sequence omitted]

SEQ ID NO: 5 - protein, 218 aa, Artificial Sequence
  Pembrolizumab, light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, Artificial Sequence
  Pembrolizumab, VH-CDR1
NYYMY

SEQ ID NO: 7 - protein, 17 aa, Artificial Sequence
  PEMBROLIZUMAB, VH-CDR2
GINPSNGGTNFNEKFKN

SEQ ID NO: 8 - protein, 11 aa, Artificial Sequence
  Pembrolizumab, VH-CDR3
RDYRFDMGFDY

SEQ ID NO: 9 - protein, 120 aa, Artificial Sequence [sequence omitted]

SEQ ID NO: 10 - protein, 447 aa, Artificial Sequence
  Pembrolizumab, heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 14 aa, Artificial Sequence
  Anti-ILT4, VL-CDR1
TGSSSNIGAGYDVH

SEQ ID NO: 12 - protein, 7 aa, Artificial Sequence
  Anti-ILT4, VL-CDR2
GDSNRPS

SEQ ID NO: 13 - protein, 11 aa, Artificial Sequence
  Anti-ILT4, VL-CDR3
QSFDNSLSAYV

SEQ ID NO: 14 - protein, 111 aa, Artificial Sequence
  Anti-ILT4, VL
ESVLTQPPSVSGAPGQRVTISCTGSSSNIGAGYDVHWYQQLPGTAPKLLIYGDSNRPSGVPDRFSVSKSGASASLAITGLQAEDEADYYCQSFDNSLSAYVFGGGTQLTVL

SEQ ID NO: 15 - protein, 217 aa, Artificial Sequence
  Anti-ILT4, light chain
ESVLTQPPSVSGAPGQRVTISCTGSSSNIGAGYDVHWYQQLPGTAPKLLIYGDSNRPSGVPDRFSVSKSGASASLAITGLQAEDEADYYCQSFDNSLSAYVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 16 - protein, 5 aa, Artificial Sequence
  Anti-ILT4, VH-CDR1
GYYWS

SEQ ID NO: 17 - protein, 16 aa, Artificial Sequence
  Anti-ILT4, VH-CDR2
EINHAGSTNYNPSLKS

SEQ ID NO: 18 - protein, 13 aa, Artificial Sequence
  Anti-ILT4, VH-CDR3
LPTRWVTTRYFDL

SEQ ID NO: 19 - protein, 121 aa, Artificial Sequence
  Anti-ILT4, VH
EVQLQQWGAGLLKPSETLSLTCAVYGGSFSGYYWSWIRQPPGKGLEWIGEINHAGSTNYNPSLKSRVTISVDTSKNQFSLKLSSVTAADTAVYYCARLPTRWVTTRYFDLWGRGTLVTVSS

SEQ ID NO: 20 - protein, 448 aa, Artificial Sequence
  Anti-ILT4, heavy chain
EVQLQQWGAGLLKPSETLSLTCAVYGGSFSGYYWSWIRQPPGKGLEWIGEINHAGSTNYNPSLKSRVTISVDTSKNQFSLKLSSVTAADTAVYYCARLPTRWVTTRYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 7 aa, Artificial Sequence
  Anti-ILT4, VL-CDR2 consensus sequence
  MISC_FEATURE (2)..(2): X1 is N, Q, E or D
  MISC_FEATURE (3)..(3): X2 is S or A
GXXNRPS

SEQ ID NO: 22 - protein, 16 aa, Artificial Sequence
  Anti-ILT4 antibody, VH-CDR2 consensus sequence
  MISC_FEATURE (5)..(5): X is S or A
EINHXGSTNYNPSLKS